(19) United States
(12) Patent Application Publication
(10) Pub. No.: US 2026/0191697 A1
(43) Pub. Date: Jul. 9, 2026

(54) MOISTURE-RESPONSIVE COMPOSITES

(71) Applicant: KIMBERLY-CLARK WORLDWIDE, INC., Neenah, WI (US)

(72) Inventors: Peiguang ZHOU, Suwanee, GA (US); Wing-Chak NG, Roswell, GA (US); Richmond R. COHEN, Appleton, WI (US); Uyen T. LAM, Menasha, WI (US)

(21) Appl. No.: 19/134,058

(22) PCT Filed: Dec. 18, 2023
(86) PCT No.: PCT/US2023/084586

§ 371 Date: May 29, 2025

Related U.S. Application Data
(60) Provisional application No. 63/477,999, filed on Dec. 30, 2022.

(51) Int. Cl. A61F 13/42; A61F 13/49; A61L 15/22; A61L 15/56

(52) U.S. Cl. CPC A61F 13/42; A61F 13/49; A61L 15/22; A61L 15/56; A61F 2013/423; A61F 2013/425; A61F 2013/426

(57) ABSTRACT

Disclosed herein are moisture-responsive composites capable of undergoing a shape change upon exposure to moisture. The composites include a moisture-responsive layer that includes a moisture-responsive polymer system. The moisture-responsive layer is laminated to a backing layer. Upon exposure to moisture, the moisture-responsive composite bends from a first position to a second position.

### CROSS-REFERENCE TO RELATED APPLICATION

This application claims the benefit of U.S. Provisional Application 63/477,999, filed Dec. 30, 2022, the entire contents of which are incorporated herein by reference.

### BACKGROUND

Elastic films and elastic laminates are widely used in personal care products, such as diapers and training pants. However, elastic materials are often difficult to integrate into automated production sequences. For example, the tension of the elastics creates challenges in cutting and other converting processes, which can slow down manufacturing and create waste.

Accordingly, there remains a need in the art for materials better suited for high-speed conversion processes that remain capable of providing certain benefits of elastic material in personal care products.

### SUMMARY OF THE DISCLOSURE

Disclosed herein are moisture-responsive composites capable of undergoing a shape change upon exposure to moisture. The composites include a moisture-responsive layer laminated to a backing layer. In certain implementations, the moisture-responsive layer is comprised of a moisture-responsive polymer system, which may be formed, for example, of at least one hydrophilic polymer and at least elastomer. The components of the moisture-responsive polymer system may be coextruded to provide a polymer film, which may be stretched in one or more directions during or after the extrusion to define the moisture-responsive layer. The moisture-responsive layer may be laminated to the backing layer, which in certain implementations may be formed from a material that does not include the moisture-responsive polymer system of the moisture-responsive layer.

When the moisture-responsive composite is exposed to moisture, a shape change is induced in the composite. In some implementations, the moisture-responsive composite is in a substantially flat configuration prior to exposure to moisture and bends (e.g., into a semi-ring-like shape) following exposure to moisture.

### BRIEF DESCRIPTION OF THE FIGURES

FIG. 1 is a side-perspective view of an absorbent article, such as a diaper, in a fastened condition according to one implementation.

FIG. 2 is a top plan view of the absorbent article of FIG. 2 in a stretched, laid flat, unfastened condition.

FIG. 3 is a front perspective view of an absorbent article, such as a pant, according to another implementation.

FIG. 4 is a top plan view of the absorbent article of FIG. 4 in a stretched, laid flat condition.

FIG. 5 is a front perspective cross-sectional view taken along line 5-5 from FIG. 2, with the absorbent article in a relaxed configuration.

FIG. 6 depicts the action of moisture on a moisture-responsive composite (100). Saline solution was dropped on the surface of the moisture-responsive polymer system (102) (FIG. 6A) and bending commenced within 15 seconds. The remainder of FIG. 6 depicts the composite after 30 seconds (FIG. 6B), 60 seconds (FIG. 6C), 80 seconds (FIG. 6D), 100 seconds (FIG. 6E), and 120 seconds (FIG. 6F). Bending accelerated from 50-60 sec and completed bending in 2 min (FIG. 6C-FIG. 6F).

FIG. 7 depicts the movement of the composite (200) under the action of atmospheric motion: at the time of creation (FIG. 7A), after two days (FIG. 7B), and after one week (FIG. 7C).

FIG. 8 depicts an x-ray diffraction of the moisture-responsive polymer system prior to exposure to water, after exposure to water, and after drying.

FIG. 9 depicts a load vs. strain curve for stretching to 300% the original length of a 60 wt. % PEO, 40% G1645/Vistamaxx 6102 (1:1 ratio) film.

FIG. 10 depicts a load vs. extension curve showing the multicycle elastic behavior of an activated pseudo plastic containing 60 wt. % PEO, 40% G1645/Vistamaxx 6102 (1:1 ratio).

FIG. 11 depicts a load vs. strain curve showing the elastic tension of 100% Kraton D1161 with around 500 g force at 300% strain.

FIG. 12 depicts a load vs. extension curve showing the pseudo elasticity of a 60 wt. % PEO, 40 wt. % D1161 moisture-responsive polymer system, indicating a stretching force around 2200 g.

FIG. 13 depicts a load vs. strain curve showing the elastic tension after the film is activated by contact with saline.

FIG. 14 depicts a load vs. extension curve for stretching to stop at 300% for the film with 60 wt. % PEO, 40 wt. % (D1161/Vistamaxx=1:1).

FIG. 15 depicts a load vs. strain curve showing multicycle testing after activation in saline for the 60 wt. % PEO, 40 wt. % (D1161/Vistamaxx=1:1)

FIG. 16 depicts a load vs. strain curve for stretching to stop at 300% for a film containing 60 wt. % PEO, 40 wt. % (80 wt. % D1161, 15 wt. % CaCO₃, 5 wt. % PE (Dowlox 2407)).

FIG. 17 depicts a load vs. strain curve showing elastic testing after activation in saline for the film containing 60 wt. % PEO, 40 wt. % (80 wt. % D1161, 15 wt. % CaCO₃, 5 wt. % PE (Dowlox 2407)).

FIG. 18 depicts a load vs. extension curve for a film containing 60 wt. % PEO, 40 wt. % (Kraton D1161/Vistamaxx 6102 (1:1 ratio), showing the dimensional stability of the moisture-responsive polymer system.

FIG. 19 depicts a load vs. strain curve for a film containing 60 wt. % PEO, 40 wt. % (Kraton D1161/Vistamaxx 6102 (1:1 ratio), showing the elasticity of the activated moisture-responsive polymer system.

FIG. 20 depicts a DSC analysis of 100% PEO.

FIG. 21 depicts a DSC analysis of 60 wt. % PEO, 40 wt. % K/V (1:1 ratio).

### DEFINITIONS

Disclosed are components that can be used to perform the disclosed methods and systems. These and other components are disclosed herein. It should be understood that when combinations, subsets, interactions, groups, etc. of these components are disclosed, while specific reference of each various individual and collective combinations and permutation of these may not be explicitly disclosed, each is specifically contemplated and described herein for all methods and systems. This applies to all aspects of this application, including, but not limited to, steps in disclosed methods. Thus, if there are a variety of additional steps that can be performed it is understood that each of these additional steps can be performed with any specific implementation or combination of implementations of the disclosed methods.

As used in the specification and the appended claims, the singular forms “a,” “an” and “the” include plural referents unless the context clearly dictates otherwise. Ranges may be expressed herein as from “about” one particular value and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

As used herein, an article or system is “moisture-responsive” if it is capable of undergoing a physical change upon exposure to water. In some implementations, the physical change is a change in shape, for example a curling, curving, or bending.

As used herein, the term “molecular weight Mw” refers to weight average molecular weight. Weight average molecular weight may also be designated with the abbreviation “MW_{w}.”

As used herein, the term “molecular weight Mn” refers to number average molecular weight. Number average molecular weight may also be designated with the abbreviation “MWₙ.”

As used herein, “polyethylene glycol,” “poly(ethylene glycol),” “polyethylene oxide,” “poly(ethylene oxide),” “PEG,” and “PEO” each interchangeably refer to a polymer having the formula H—[OCH₂CH₂]ₙ—OH, wherein the number of monomer units is given by the average molecular weight of the polymer.

As used herein, “machine direction” refers to the direction an extruded polymer film exits the extruding machine. In the context of a polymer film, the machine direction lies along an axis that is parallel to the film and oriented in the direction the film exited the extruding machine.

As used herein, “transverse direction” and “cross direction” (which may be used interchangeable) refers to the direction that lies along an axis that is parallel to the film and oriented in the direction that is perpendicular to the machine direction.

As used herein, “uniaxially stretched” refers to a polymer film that has been stretched in a single direction (e.g., in the machine direction). Films that are stretched by applying opposing forces opposite each other, and films that are stretched by applying a force to one end of the film while holding the opposite end in place, are both considered uniaxially stretched.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular implementations only and is not intended to be limiting.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

### Moisture-Responsive Composites

Disclosed herein are composites capable of undergoing a shape change upon exposure to moisture (e.g., water). In various implementations, the composite is a laminate having at least one moisture-responsive layer and a backing layer. The moisture-responsive layer includes a moisture-responsive polymer system (e.g., one or more hydrophilic polymers and one or more elastomers). The moisture-responsive layer(s) and backing layer are laminated together such that they remain attached to one another even when wet, thereby forming a moisture-responsive composite. The resulting moisture-responsive composite may be a laminate having a width, length, and thickness.

Upon exposure to moisture, the moisture-responsive composite undergoes a shape change. The shape change can be a bending, curling, or coiling in one or more directions. For example, in certain implementations the shape change can be up to a 360° bending of the moisture-responsive composite. In certain implementations, the shape change be up to a 180° bending of the moisture-responsive composite. In various implementations, the moisture-responsive composite includes at least one elastomeric polymer under tension, such that exposure to moisture releases the stored tension and causes the moisture-responsive composite to change its shape.

In various implementations, the moisture-responsive layer and backing layer may be distinguished, for example, by their respective modulus. Generally, the backing layer will have a higher modulus than the moisture-responsive layer. For example, in some implementations, the modulus of the backing layer will be at least 110%, at least 125%, at least 150%, at least 200%, at least 250%, at least 300%, at least 400%, or at least 500% the modulus of the moisture-responsive layer.

In some implementations, the backing layer and moisture-responsive layer may be distinguished according to their elastic modulus. Generally, the backing layer will have a higher elastic modulus than the moisture-responsive layer. In some implementations, the elastic modulus of the backing layer will be at least 110%, at least 125%, at least 150%, at least 200%, at least 250%, at least 300%, at least 400%, or at least 500% the elastic modulus of the moisture-responsive layer.

In some implementations, the backing layer and moisture-responsive layer may be distinguished according to their Young's modulus. Generally, the backing layer will have a higher Young's modulus than the moisture-responsive layer. In some implementations, the Young's modulus of the backing layer will be at least 110%, at least 125%, at least 150%, at least 200%, at least 250%, at least 300%, at least 400%, or at least 500% the Young's modulus of the moisture-responsive layer.

In some implementations, the backing layer and moisture-responsive layer may be distinguished according to their flexural modulus. Generally, the backing layer will have a higher flexural modulus than the moisture-responsive layer. In some implementations, the flexural modulus of the backing layer will be at least 110%, at least 125%, at least 150%, at least 200%, at least 250%, at least 300%, at least 400%, or at least 500% the flexural modulus of the moisture-responsive layer.

In some implementations, the backing layer and moisture-responsive layer may be distinguished according to their bending stiffness. Generally, the backing layer will have a higher bending stiffness than the moisture-responsive layer. In some implementations, the bending stiffness of the backing layer will be at least 110%, at least 125%, at least 150%, at least 200%, at least 250%, at least 300%, at least 400%, or at least 500% the bending stiffness of the moisture-responsive layer.

In various implementations, the moisture-responsive layer may be laminated to the backing layer using an adhesive or autogeneously (e.g., fusion and/or self-adhesion of the fibers without an applied external adhesive). Autogenous bonding, for instance, may be achieved through contact of the layers while they are semi-molten or tacky, or simply by blending a tackifying resin and/or solvent with the materials used to form the backing layer or moisture-responsive polymer layer. Suitable autogenous bonding techniques may include ultrasonic bonding, thermal bonding, pressure bonding through-air bonding, calendar bonding, and so forth, provided that the resulting laminate does not decouple in the presence of moisture. Suitable adhesives may include, for instance, hot melt adhesives, pressure-sensitive adhesives, and so forth. When utilized, the adhesive may be applied as a uniform layer, a patterned layer, a sprayed pattern, or any of separate lines, swirls or dots.

### Moisture-Responsive Layer

In various implementations, the moisture-responsive layer includes one or more moisture-responsive polymer systems. In some implementations, the moisture-responsive polymer system includes one or more hydrophilic polymers and one or more elastomers.

### Hydrophilic Polymers (of the Moisture-Responsive Polymer System)

Exemplary hydrophilic polymers include poly(vinyl pyrrolidone), poly(hydroxyethyl (meth)acrylate, poly(hydroxypropyl (meth)acrylate, poly(meth)acrylic acid, poly(vinyl pyridine), poly(meth)acrylamide, poly(vinyl acetate), poly(vinyl alcohol), poly(ethylene oxide), copolymers thereof, and mixtures thereof. In certain implementations, the hydrophilic polymer includes a poly(ethylene oxide) homopolymer (i.e., “PEO”), a poly(ethylene oxide-co-propylene oxide) copolymer (i.e., “PEO-PPO”), or a combination thereof. In some implementations the poly(ethylene oxide-co-propylene oxide) copolymer is a block copolymer, for example PPO-PEO-PPO or PEO-PPO-PEO. In certain implementations, the hydrophilic polymer includes two different poly(ethylene oxide) homopolymers, wherein the two polymers are distinguished in terms of molecular weight.

In some implementations, the hydrophilic polymer can have an average molecular weight (MW_{w}) from 10,000-1,000,000 Da, from 10,000-500,000 Da, from 50,000-500,000 Da, from 50,000-250,000 Da, from 50,000-1000,000, from 100,000-500,000 Da, from 100,000-300,000 Da, from 200,000-400,000 Da, from 150,000-300,000 Da, or from 250,000-500,000 Da. Preferably the hydrophilic polymer is PEO. In certain implementations, the hydrophilic polymer includes two different poly(ethylene oxide) homopolymers, wherein the first PEO has a MW_{w} from 10,000-300,000 Da, from 25,000-300,000, from 50,000-300,000 Da, from 100,000-300,000 Da, from 200,000-300,000 Da, from 100,000-200,000 Da, or from 50,000-150,000 Da, and the second PEO has a MW_{w} from 200,000-750,000 Da, from 300,000-750,000 Da, from 400,000-750,000 Da, from 500,000-750,000 Da, from 200,000-500,000 Da, from 300,000-500,000 Da, from 200,000-400,000 Da, from 300,000-400,000 Da, or from 400,000-500,000 Da.

In some implementations, the hydrophilic polymer can have an average molecular weight (MWₙ) from 10,000-1,000,000 Da, from 10,000-500,000 Da, from 50,000-500,000 Da, from 50,000-250,000 Da, from 50,000-1000,000, from 100,000-500,000 Da, from 100,000-300,000 Da, from 200,000-400,000 Da, from 150,000-300,000 Da, or from 250,000-500,000 Da. Preferably the hydrophilic polymer is PEO. In certain implementations, the hydrophilic polymer includes two different poly(ethylene oxide) homopolymers, wherein the first PEO has a MWₙ from 10,000-300,000 Da, from 25,000-300,000, from 50,000-300,000 Da, from 100,000-300,000 Da, from 200,000-300,000 Da, from 100,000-200,000 Da, or from 50,000-150,000 Da, and the second PEO has a MWₙ from 200,000-750,000 Da, from 300,000-750,000 Da, from 400,000-750,000 Da, from 500,000-750,000 Da, from 200,000-500,000 Da, from 300,000-500,000 Da, from 200,000-400,000 Da, from 300,000-400,000 Da, or from 400,000-500,000 Da.

In some implementations, the hydrophilic polymer is a water-soluble polymer. In some implementations the hydrophilic polymer is a water-dispersible polymer. In some implementations, the hydrophilic polymer has a solubility in water, at 23° C., of at least 0.1 g/mL, at least 0.5 g/mL, at least 1 g/mL, at least 2.5 g/mL, at least 5 g/mL, or at least 10 g/mL.

### Elastomers (of the Moisture-Responsive Polymer System)

In some implementations, the elastomer is a polyolefin elastomer. The polyolefin may have a melting temperature of from about 100° C. to about 220° C., in some implementations from about 120° C. to about 200° C., and in some implementations, from about 140° C. to about 180° C. The melting temperature may be determined using differential scanning calorimetry (“DSC”) in accordance with ASTM D-3417. Suitable polyolefins may, for instance, include ethylene polymers (e.g., low density polyethylene (“LDPE”), high density polyethylene (“HDPE”), linear low density polyethylene (“LLDPE”), etc.), propylene homopolymers (e.g., syndiotactic, atactic, isotactic, etc.), propylene copolymers, and so forth. In one particular implementation, the polymer is a propylene polymer, such as propylene homopolymer or a copolymer of propylene. The propylene polymer may, for instance, be formed from a substantially isotactic polypropylene homopolymer or a copolymer containing equal to or less than about 10 wt. % of other monomers, i.e., at least about 90% by weight propylene. Such homopolymers may have a melting point of from about 140° C. to about 170° C.

In some implementations, the polyolefin may be a copolymer of ethylene or propylene with another α-olefin, such as a C₃-C₂₀ α-olefin or C₃-C₁₂ α-olefin. Specific examples of suitable α-olefins include 1-butene; 3-methyl-1-butene; 3,3-dimethyl-1-butene; 1-pentene; 1-pentene with one or more methyl, ethyl or propyl substituents; 1-hexene with one or more methyl, ethyl or propyl substituents; 1-heptene with one or more methyl, ethyl or propyl substituents; 1-octene with one or more methyl, ethyl or propyl substituents; 1-nonene with one or more methyl, ethyl or propyl substituents; ethyl, methyl or dimethyl-substituted 1-decene; 1-dodecene; and styrene. Particularly desired α-olefin comonomers are 1-butene, 1-hexene and 1-octene. The ethylene or propylene content of such copolymers may be from about 60 mole % to about 99 mole %, in some implementations from about 80 mole % to about 98.5 mole %, and in some implementations, from about 87 mole % to about 97.5 mole %. The α-olefin content may likewise range from about 1 mole % to about 40 mole %, in some implementations from about 1.5 mole % to about 15 mole %, and in some implementations, from about 2.5 mole % to about 13 mole %.

In some implementations the elastomer includes ethylene-based copolymers available under the designation EXACT™ from ExxonMobil Chemical Company of Houston, Texas, or ENGAGE™, AFFINITY™, DOWLEX™ (LLDPE) and ATTANE™ (ULDPE) from Dow Chemical Company of Midland, Mich. Other suitable ethylene polymers are described in U.S. Pat. No. 4,937,299 to Ewen et al.; U.S. Pat. No. 5,218,071 to Tsutsui et al.; U.S. Pat. No. 5,272,236 to Lai. et al.; and U.S. Pat. No. 5,278,272 to Lai, et al. In some implementations the elastomer includes propylene-based copolymers available under the designation VISTAMAXX™ from ExxonMobil Chemical Co. of Houston, Tex.; FINA™ (e.g., 8573) from Atofina Chemicals of Feluy, Belgium; TAFMER™ available from Mitsui Petrochemical Industries; and VERSIFY™ available from Dow Chemical Co. of Midland, Mich. Suitable polypropylene homopolymers may include Exxon Mobil 3155 polypropylene, Exxon Mobil Achieve™ resins, and Total M3661 PP resin. Other examples of suitable propylene polymers are described in U.S. Pat. No. 6,500,563 to Datta, et al.; U.S. Pat. No. 5,539,056 to Yana, et al.; and U.S. Pat. No. 5,596,052 to Resconi, et al.

In some implementations, the elastomer is a styrene block copolymer, a butadiene block copolymer, a hydrogenated butadiene block copolymer, an isoprene block copolymer, a hydrogenated isoprene block copolymer, non-crystalline ethylene/α-olefin random copolymer, low-crystalline ethylene/α-olefin random copolymer, propylene/ethylene/α-olefin random copolymer, or combination thereof.

In some implementations, the elastomer includes a polystyrene-polybutadiene-polystyrene block copolymer (SBS), polystyrene-polyisoprene-polystyrene block copolymer (SIS), polystyrene-poly/ethylene/butylene-polystyrene block copolymer (SEBS), polystyrene-poly/ethylene/propylene-polystyrene block copolymer, or a combination thereof.

In certain implementations, the elastomer includes an ethylene/propylene random copolymer, ethylene/1-butene random copolymer, propylene/1-butene random copolymer, or combination thereof.

In some implementations, the elastomer includes ethylene/α-olefin copolymer, propylene/α-olefin copolymer, styrene-olefin copolymer, or a combination thereof. In certain implementations, the elastomer includes poly(ethylene-butene), poly(ethylene-hexene), poly(ethylene-octene), poly(ethylene-propylene), poly(styrene-butadiene-styrene), poly(styrene-isoprene-styrene), poly(styrene-ethylene-butylene-styrene), poly(ester-ether), poly(ether-amide), poly(ethylene-vinylacetate), poly(ethylene-methylacrylate), poly(ethylene-acrylic acid), poly(ethylene butylacrylate), polyurethane, poly(ethylene-propylene-diene), ethylene-propylene rubber, or a combination thereof.

In some implementations, the elastomer includes a polymer available from Kraton Polymers LLC of Houston, Tex. under the trade name KRATON™ (herein “Kraton”). Kraton polymers include styrene-diene block copolymers, such as styrene-butadiene, styrene-isoprene, styrene-butadiene-styrene, styrene-isoprene-styrene, and styrene-isoprene/butadiene-styrene. Kraton polymers also include styrene-olefin block copolymers formed by selective hydrogenation of styrene-diene block copolymers. Examples of such styrene-olefin block copolymers include styrene-(ethylene-butylene), styrene-(ethylene-propylene), styrene-(ethylene-butylene)-styrene, styrene-(ethylene-propylene)-styrene, styrene-(ethylene-butylene)-styrene-(ethylene-butylene), styrene-(ethylene-propylene)-styrene-(ethylene-propylene), and styrene-ethylene-(ethylene-propylene)-styrene. These styrenic block copolymers may have a linear, radial, or star-shaped molecular configuration. Specific Kraton block copolymers include those sold under the brand names G 1652, G 1657, G 1730, MD6673, and MD6973. Various suitable styrenic block copolymers are described in U.S. Pat. No. 4,663,220 to Wisneski, et al., U.S. Pat. No. 4,323,534 to DesMarais, U.S. Pat. No. 4,834,738 to Kielpikowski, et al., U.S. Pat. No. 5,093,422 to Himes, and U.S. Pat. No. 5,304,599 to Himes, which are hereby incorporated in their entirety by reference thereto for all purposes. Other commercially available block copolymers include the S-EP-S elastomeric copolymers available from Kuraray Company, Ltd. of Okayama, Japan, under the trade designation SEPTON™. Still other suitable copolymers include the S-I-S and S-B-S elastomeric copolymers, which can be available from Dexco Polymers of Houston, Tex. or TSRC Company of Taiwan under the trade designation VECTOR™. Also suitable are polymers composed of an A-B-A-B tetrablock copolymer, such as discussed in U.S. Pat. No. 5,332,613 to Taylor, et al., which is incorporated herein in its entirety by reference thereto for all purposes. An example of such a tetrablock copolymer is a styrene-poly(ethylene-propylene)-styrene-poly(ethylene-propylene) (“S-EP-S-EP”) block copolymer.

### Moisture-Responsive Polymer Systems

The one or more hydrophilic polymers and one or more elastomers (e.g., those described herein) may be combined in a variety of different weight ratios to form the moisture-responsive polymer system. In those implementations having more than one hydrophilic polymer and/or more than one elastomer, the ratios given below refer to the total weight of all hydrophilic polymers and the total weight of all elastomers.

In some implementations, the moisture-responsive polymer system includes no more than 75 wt. % hydrophilic polymer, e.g., from 25:75 to 75:25, from 30:70 to 75:25, from 35:65 to 75:25, from 40:60 to 75:25, from 45:55 to 75:25, from 50:50 to 75:25, from 55:45 to 75:25, from 60:40 to 75:25, or from 65:35 to 75:25.

In some implementations, the moisture-responsive polymer system includes no more than 70 wt. % hydrophilic polymer, e.g., from 25:75 to 70:30, from 30:70 to 70:30, from 35:65 to 70:30, from 40:60 to 70:30, from 45:55 to 70:30, from 50:50 to 70:30, from 55:45 to 70:30, from 60:40 to 70:30, or from 65:35 to 70:30.

In some implementations, the moisture-responsive polymer system includes no more than 65 wt. % hydrophilic polymer, e.g., from 25:75 to 65:35, from 30:70 to 65:35, from 35:65 to 65:35, from 40:60 to 65:35, from 45:55 to 65:35, from 50:50 to 65:35, from 55:45 to 65:35, or from 60:30 to 65:35.

In some implementations, the moisture-responsive polymer system includes no more than 60 wt. % hydrophilic polymer, e.g., from 25:75 to 60:40, from 30:70 to 60:40, from 35:65 to 60:40, from 40:60 to 60:40, from 45:55 to 60:40, from 50:50 to 60:40, or from 55:45 to 60:40.

In some implementations, the moisture-responsive polymer system includes no more than 55 wt. % hydrophilic polymer, e.g., from 25:75 to 55:45, from 30:70 to 55:45, from 35:65 to 55:45, from 40:60 to 55:45, from 45:55 to 55:45, or from 50:50 to 55:45.

In some implementations, the moisture-responsive polymer system includes no more than 50 wt. % hydrophilic polymer, e.g., from 25:75 to 50:50, from 30:70 to 50:50, from 35:65 to 50:50, from 40:60 to 50:50, or from 45:55 to 50:50.

In some implementations, the moisture-responsive polymer system includes no more than 45 wt. % hydrophilic polymer, e.g., from 25:75 to 45:55, from 30:70 to 45:55, from 35:65 to 45:55, or from 40:60 to 45:55.

In some implementations, the moisture-responsive polymer system includes no more than 40 wt. % hydrophilic polymer, e.g., from 25:75 to 40:60, from 30:70 to 40:60, or from 35:65 to 40:60.

In some implementations, the moisture-responsive polymer system includes no more than 35 wt. % hydrophilic polymer, e.g., from 25:75 to 35:65, or from 30:70 to 35:65.

The moisture-responsive polymer system may be provided in a variety of different basis weights. In some implementations, the moisture-responsive polymer system has a basis weight between about 1-500, about 1-250, about 5-250, about 10-250, about 10-100, about 10-50, about 10-25, about 1-10, about 5-25, about 25-50, about 25-75, about 50-100, about 75-125, about 100-150, about 50-150, about 100-250, or about 250-500 grams per square meter.

The moisture-responsive layer may be provided in a variety of different thicknesses. In some implementations, the moisture-responsive layer has a thickness from, from 0.01-1 mm, from 0.01-0.5 mm, from 0.01-0.25 mm, from 0.01-0.1 mm, from 0.05-1 mm, from 0.1-1 mm, from 0.5-1.5 mm, from 0.5-2.5 mm, from 1-2.5 mm, or from 1-15 mm.

### Manufacture of Moisture-Responsive Polymer Systems

The moisture-responsive polymer system may be prepared by extruding a mixture of hydrophilic polymer(s) and elastomer(s). In some implementations, the mixture is a pre-compounded mixture, while in other implementations the mixture is not a pre-compounded mixture. In some implementations, the moisture-responsive polymer system is extruded through a single screw extruder, while in other implementations, the moisture-responsive polymer system is extruded through a twin-screw extruder. The extruded moisture-responsive polymer system will have a machine direction, which is the direction the moisture-responsive polymer system exits the extruder, as well as a transverse, or cross direction, which is perpendicular to the machine direction.

In some implementations, the extruded moisture-responsive polymer system is stretched in one or more directions to provide an elongated moisture-responsive polymer system. The moisture-responsive polymer system may be stretched in-line as it is being formed, it may be stretched following extrusion and before, during, and/or after lamination with the backing layer. The extruded moisture-responsive polymer system may be stretched to have a certain stretch ratio. The stretch ratio may be determined by dividing the length of the moisture-responsive polymer system after stretching in a certain direction by its length in the same direction prior to stretching. By definition, an unstretched moisture-responsive polymer system has a stretch ratio of 1. The stretch ratio may also be approximately the same as the draw ratio, which may be determined by dividing the linear speed of the moisture-responsive polymer system upon stretching (e.g., speed of the nip rolls) by the linear speed at which the moisture-responsive polymer system is formed (e.g., speed of casting rolls or blown nip rolls).

In certain implementations, the extruded moisture-responsive polymer system is stretched in the machine direction. In some implementations, the extruded moisture-responsive polymer system is stretched in the cross direction. In some implementations, the extruded moisture-responsive polymer system is stretched at an angle between the machine and traverse directions. In certain implementations, taking the machine direction as the x-axis (angle=0°) and the cross direction as the y-axis (angle=90°), the film may be stretched for instance at an angle that is 5°, 10°, 15°, 20°, 25°, 30°, 35°, 40°, 45°, 50°, 55°, 60°, 65°, 70°, 75°, 80°, or 85°. When the moisture-responsive polymer system is only stretched in one direction, it can be designated a uniaxially stretched moisture-responsive polymer system. A moisture-responsive polymer system stretched in two directions (e.g., in the machine direction and the transverse direction or an angle between the machine direction and transverse direction) can be designated a biaxially stretched moisture-responsive polymer system.

In some implementations, the elongated moisture-responsive polymer system has a stretch ratio from 1.1-20, 2-15, 2-10, 2-8, 2-6, 2-4, 3-6, 3-6, 4-6, 4-7, 5-7, 5-8, 5-10, 5-15, or 10-20. In some implementations, the elongated moisture-responsive polymer system is stretched to a length that is 2-10 times, 2-8 times, 2-6 times, 2-4 times, 3-6 times, 3-6 times, 4-6 times, 4-7 times, 5-7 times, 5-8 times, or 5-10 times the length of the moisture-responsive polymer system prior to stretching. In certain preferred implementations, the moisture-responsive polymer system is stretched to a length that is about 300% the length of the unstretched system.

In some implementations, the elongated moisture-responsive polymer system has a basis weight (gsm) that is no more than 90%, no more than 80%, no more than 70%, no more than 60%, no more than 50%, no more than 40%, no more than 30%, or no more than 20% the basis weight of the moisture-responsive polymer system prior to stretching. In certain preferred implementations, the moisture-responsive polymer system is stretched such that it has a basis weight that is about 40-60% the basis weight of the unstretched moisture-responsive polymer system.

In some implementations, the elongated moisture-responsive polymer system has a width that is no more than 90%, no more than 80%, no more than 70%, no more than 60%, no more than 50%, no more than 40%, no more than 30%, or no more than 20% the width of the moisture-responsive polymer system prior to stretching.

In certain implementations, the elongated moisture-responsive polymer system may include the hydrophilic polymer(s) in a crystalline state. In some implementations, when the moisture-responsive polymer system is in the stretched or elongated state, the hydrophilic polymer has a degree of crystallinity of at least 25%, at least 30%, at least 40%, at least 50%, at least 60%, at least 70%, at least 80%, or at least 90%. The degree of crystallinity may be determined using DSC. The hydrophilic polymer may be further characterized by rapid conversion to the amorphous form when the moisture-responsive polymer system is submerged in water. For example, the crystalline hydrophilic polymer present in the elongated moisture-responsive polymer system may convert to the amorphous form within 120 seconds, 100 seconds, 80 seconds, 60 seconds, 50 seconds, 40 seconds, 30 seconds, 20 seconds, or at least 10 seconds when submerged in water. As used herein, a polymer is considered converted to the amorphous form when at least 90% of the hydrophilic polymer is in the amorphous form.

The moisture-responsive polymer system may be stretched according to techniques known in the art. The moisture-responsive polymer system may be stretched at a temperature from about 15° C. to about 50° C., in some implementations from about 25° C. to about 40° C., and in some implementations, from about 30° C. to about 40° C. In some implementations, the moisture-responsive polymer system is “cold drawn”, i.e., stretched without the application of external heat (e.g., heated rolls). In certain implementations, the stretching is performed in an atmosphere having a temperature from 20-30° C., 20-50° C. or from 50-150° C.

The moisture-responsive polymer system can be stretched by a laboratory stretching machine, such as the Machine Direction Orientation (MDO) unit FOVU-40 from Guangzhou POTOP Experimental Analysis Instrument Co., China. Suitable laboratory stretching machines include, for example, preheating rolls, stretching rolls, and cooled annealing rolls. The moisture-responsive film may be heated, longitudinal stretched, annealing shaped, and cooled, with stretching ratio up to 10, and a final stretching speed 50 ft/min.

In some implementations, the methods for producing the film may include batch and/or continuous melt processing techniques. For example, a mixer/kneader, Banbury mixer, Farrel continuous mixer, single-screw extruder, twin-screw extruder, roll mill, etc., may be utilized to blend and melt process the materials. Examples of suitable melt processing devices may include a co-rotating, twin-screw extruder (e.g., USALAB twin-screw extruder available from Thermo Electron Corporation of Stone, England or an extruder available from Werner-Pfreiderer from Ramsey, N.J.). Such extruders may include feeding and venting ports and provide high intensity distributive and dispersive mixing. For example, the components may be fed to the same or different feeding ports of the twin-screw extruder and melt blended to form a substantially homogeneous melted mixture. If desired, other additives may also be injected into the polymer melt and/or separately fed into the extruder at a different point along its length.

In some implementations, the film can be produced in a melt-blending device. For example, the hydrophilic polymer and elastomer can be melt-blended and extruded in an extruder. The hydrophilic polymer and elastomer can be separately supplied to an extruder where they are uniformly blended. In some implementations, the film can be produced using a single-screw extruder or a twin-screw extruder (e.g., co-rotating, twin-screw extruder). Commercially available single-screw extruders suitable for producing the film include, for example, HAAKE™ Rheomex OS Single Screw Extruder from Thermo Fisher Scientific, Waltham, MA. Commercially available twin-screw extruders include, for example, a ZSK-30 extruder available from Werner & Pfleiderer Corporation of Ramsey, N.J., or a Thermo Prism™ USALAB 16 extruder available from Thermo Electron Corp., Stone, England. In some implementations, melt blending may occur at a temperature of from 50° C. to 300° C., e.g., from 75° C. to 250° C., from 100° C. to 225° C., from 120° C. to 215° C. or from 120° C. to 200° C. In some implementations, the screw speed of the extruder may be up to 200 RPM. The polymer melt from the extruder can be processed in the film die to produce the film. In some implementations, the film can be collected on one or more chill rollers.

In some implementations, the film can be produced in a two-step process including compounding and extruding the film. In this implementation, the hydrophilic polymer and elastomer can be compounded into pellets, and the pellets can be extruded to produce the film. For example, the hydrophilic polymer and elastomer can be melt blended via the twin screw extruder at an extrusion temperature to form a homogeneous polymer blend. The molten polymer blend can then be extruded through a filament die to produce a sheet. Thereafter, the extruded material may be chilled and cut into pellet form. In some implementations, the extruded material can be air-cooled on a conveyor using fans, and then cut into pellets using a pelletizing system. The compounded pellets can be melted in another twin-screw extruder at an extrusion temperature and extruded through a film die onto chill rollers.

In some implementations, the moisture-responsive polymer system may be uniaxially stretched by MDO using a multi-stretch sequence. In some implementations, the moisture-responsive polymer system is stretched under a first set of stretching conditions, and then stretched under a second set of stretching conditions, in which the first and second sets of conditions are not the same. For example, the first stretching may be conducted at a temperature higher than the second stretching, or the second stretching may be conducted at a temperature higher than the first stretching. In some implementations, the first stretching is conducted using a greater stretching force than the second stretching. In some implementations, the second stretching is conducted using a greater stretching force than the first stretching.

### Additional Components for Moisture-Responsive Layer

Besides polymers, the moisture-responsive polymer system may also contain other components, for example additional materials in the extrusion mix, as is known in the art. In one implementation, for example, the elastic composition contains a filler. Fillers are particulates or other forms of material that may be added to an extrusion mixture and that will not chemically interfere with the extruded polymer, but which may be uniformly dispersed throughout the moisture-responsive polymer system. Fillers may serve a variety of purposes, including enhancing opacity and/or breathability (i.e., fillers that are vapor-permeable and substantially liquid-impermeable). In some implementations, the filler is calcium carbonate, titanium dioxide, or a combination thereof.

Other additives may also be incorporated into the elongated moisture-responsive polymer system, such as crosslinking catalysts, pro-rad additives, melt stabilizers, processing stabilizers, heat stabilizers, light stabilizers, antioxidants, heat aging stabilizers, whitening agents, antiblocking agents, bonding agents, viscosity modifiers, etc. Suitable crosslinking catalysts, for instance, may include organic bases, carboxylic acids, and organometallic compounds such as organic titanates and complexes or carboxylates of lead, cobalt, iron, nickel, zinc, and tin (e.g., dibutyltin dilaurate, dioctyltin maleate, dibutyltin diacetate, dibutyltin dioctoate, stannous acetate, stannous octoate, lead naphthenate, zinc caprylate, cobalt naphthenate, etc.). Suitable pro-rad additives may likewise include azo compounds, organic peroxides, and polyfunctional vinyl or allyl compounds such as triallyl cyanurate, triallyl isocyanurate, pentaerythritol tetramethacrylate, glutaraldehyde, polyester acrylate oligomers (e.g., available from Sartomer under the designation CN2303), ethylene glycol dimethacrylate, diallyl maleate, dipropargyl maleate, dipropargyl monoallyl cyanurate, dicumyl peroxide, di-tert-butyl peroxide, t-butyl perbenzoate, benzoyl peroxide, cumene hydroperoxide, t-butyl peroctoate, methyl ethyl ketone peroxide, 2,5-dimethyl-2,5-di(t-butyl peroxy) hexane, lauryl peroxide, tert-butyl peracetate, azobisisobutyl nitrite, etc.

Phosphite stabilizers (e.g., IRGAFOS available from Ciba Specialty Chemicals of Terrytown, N.Y. and DOVERPHOS available from Dover Chemical Corp. of Dover, Ohio) are exemplary melt stabilizers. In addition, hindered amine stabilizers (e.g., CHIMASSORB available from Ciba Specialty Chemicals) are exemplary heat and light stabilizers. Further, hindered phenols are commonly used as an antioxidant in the production of films. Some suitable hindered phenols include those available from Ciba Specialty Chemicals under the trade name “Irganox®”, such as Irganox® 1076, 1010, or E 201. Moreover, bonding agents may also be added to the elastic composition to facilitate bonding to additional materials (e.g., nonwoven web). When employed, such additives (e.g., tackifier, antioxidant, stabilizer, crosslinking agents, pro-rad additives, etc.) may each be present in an amount from about 0.001 wt. % to about 25 wt. %, in some implementations, from about 0.005 wt. % to about 20 wt. %, and in some implementations, from 0.01 wt. % to about 15 wt. % of the elastic material.

### Backing Layer

The moisture-responsive composite's backing layer may include a variety of different materials, such as a hydrophobic material, a hydrophilic material, or a combination thereof. For example, the backing layer can include nonwoven materials, wetlaid, airlaid, spunbond, meltblown, coform, bonded-carded webs, foams, tissue, netting, scrim, woven materials, or a combination thereof. The backing layer may be defined by its basis weight prior to lamination to the moisture-responsive layer. In some implementations the backing layer has a basis weight between about 1-100 grams per square meter, for example 1-50, 1-25, 1-10, 1-5, 5-10, 5-15, 10-20, 10-25, 10-50, 25-50, 25-75, 25-100, 50-100, or 75-100 grams per square meter. In some implementations, the backing layer has a basis weight between about 1-1,000, grams per square meter, for example about 25-1,000, about 50-1,000, about 100-1,000, about 1-500, about 1-250, about 5-250, about 10-250, about 10-100, about 50-100, about 75-125, about 100-150, about 50-150, about 100-250, about 250-500, or about 500-1,000 grams per square meter.

The backing layer may be provided in a variety of different thicknesses. In some implementations, the backing layer has a thickness from, from 0.01-1 mm, from 0.01-0.5 mm, from 0.01-0.25 mm, from 0.01-0.1 mm, from 0.05-1 mm, from 0.1-1 mm, from 0.5-1.5 mm, from 0.5-2.5 mm, or from 1-2.5 mm.

### Moisture-Responsive Shape Changes

Prior to undergoing the shape change, the composite may be said to be in a first position, and upon exposure to moisture, the composite moves towards a second position, thereby changing its shape. In some implementations, the first position is substantially flat, while in other implementations the first position is a curved and/or bent shape. In certain implementations, the second position is characterized by an increased concavity along the interface of the backing and moisture-responsive layers.

In some implementations, the shape change may be defined relative to a center line extending longitudinally through the composite. For implementations in which the first position is a substantially flat position, the center line will be substantially flat with respect to the length and width of the composite as well. For implementations in which the first position has a curved or bent shape, the center line may be curved or bent to remain in plane with the composite. As the composite moves from the first position towards the second position, the radius of curvature of the center line decreases. Such a movement may be designated a curl. In certain implementations the radius of curvature decreases such that two positions along the center line, which are separated in space in the first position, and brought into closer proximity as the composite moves towards the second position. In some implementations, the separated two positions along the center line, which are separated in space in the first position are brought into contact with one another as the composite moves towards the second position.

In certain implementations, the composite can have a first end and second end along the center line. When the composite adopts a ring shape as it moves towards the second position, the first end and second end contact one another. The center line may be substantially in the same direction the elongated moisture-responsive polymer system was stretched.

The moisture sensitive composites may be included in without limitation, personal care absorbent articles and medical absorbent articles. Personal care absorbent articles include but are not limited to diapers, training pants, swim wear, absorbent underpants, child-care pants, adult incontinence products including but not limited to pads, containers, incontinence products, and urinary shields, feminine hygiene products including but not limited to sanitary napkins, menstrual pads, panty liners, panty shields, interlabials, tampons, and the like. Medical absorbent articles include medical absorbent garments, drapes, gowns, bandages, wound dressings, underpads, bed pads, face masks, and the like. Besides personal care products and medical absorbent articles, the moisture sensitive composites can also be used in a wide array of applications including but not limited to a variety of cleaning applications, clothing components, filters, athletic and recreation products, and construction and packaging uses.

### Absorbent Articles Having Moisture Sensitive Composites

Also disclosed herein are various articles of manufacture which combine the moisture sensitive composites described herein with various articles of manufacture, such as absorbent articles and/or tissue products.

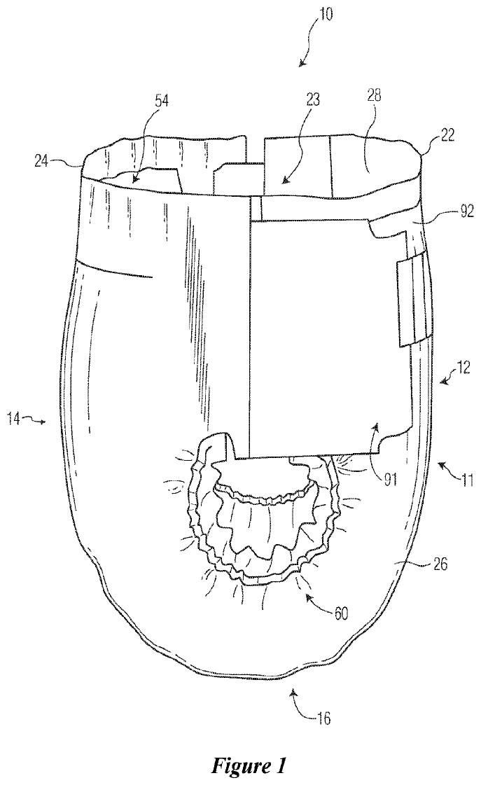

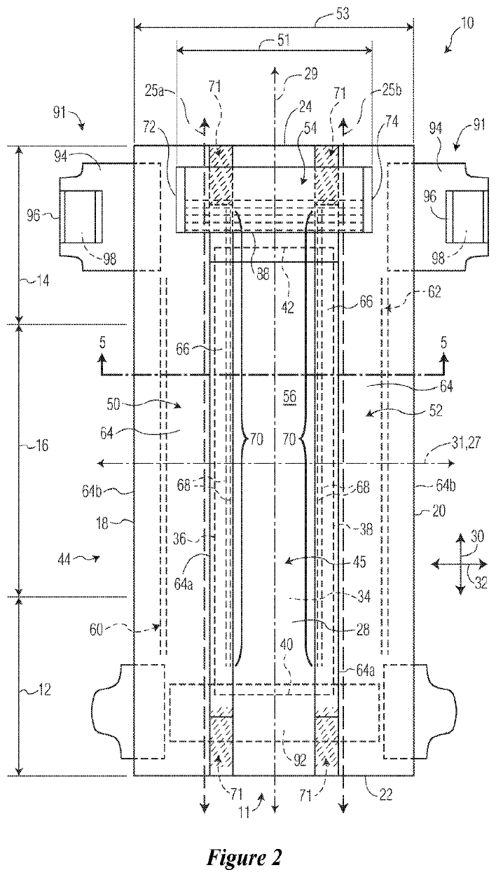

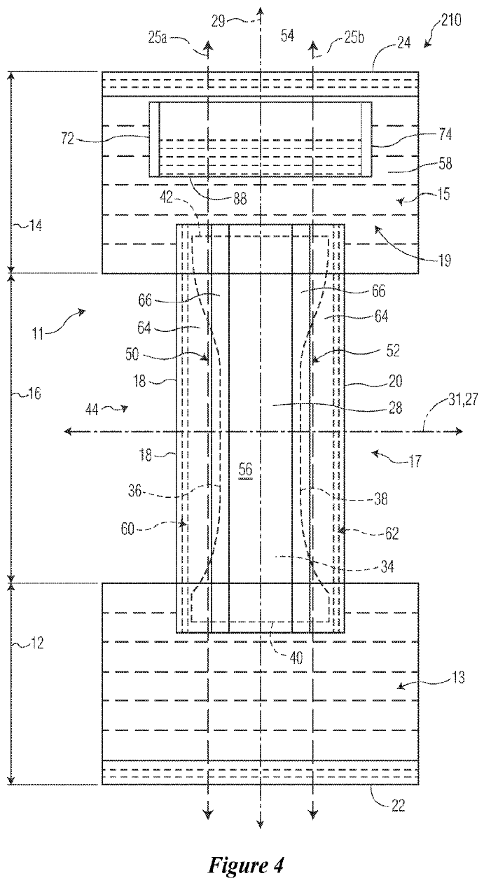

Referring to FIGS. 1 and 2, a non-limiting illustration of an absorbent article 10, for example a diaper, is illustrated. While the implementations and illustrations described herein may generally apply to absorbent articles manufactured in the product longitudinal direction, which is hereinafter called the machine direction manufacturing of a product, it should be noted that one of ordinary skill in the art could apply the information herein to absorbent articles manufactured in the latitudinal direction of the product, which hereinafter is called the cross-machine direction manufacturing of a product, without departing from the spirit and scope of the disclosure. For example, the absorbent article 210 shown in FIGS. 43 and 4 provides an implementation of an absorbent article 210 that can be manufactured in cross-machine direction manufacturing process.

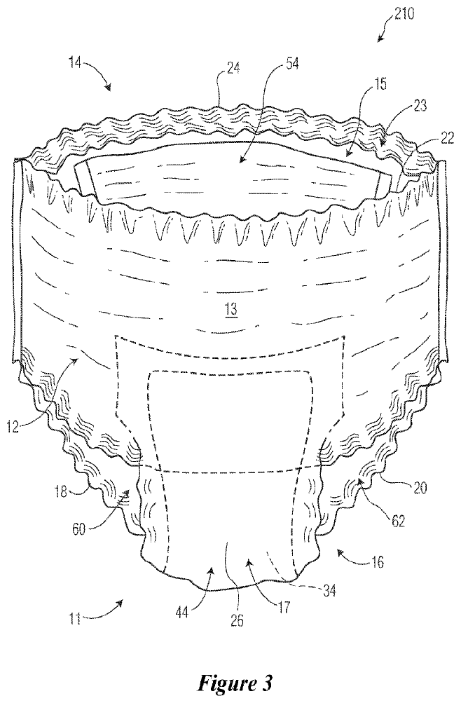

The absorbent article 10 illustrated in FIGS. 1 and 2 and the absorbent article 210 illustrated in FIGS. 3 and 4 can each include a chassis 11. The absorbent article 10, 210 can include a front waist region 12, a rear waist region 14, and a crotch region 16 disposed between the front waist region 12 and the rear waist region 14 and interconnecting the front and rear waist regions, 12, 14, respectively. The front waist region 12 can be referred to as the front-end region, the rear waist region 14 can be referred to as the rear-end region, and the crotch region 16 can be referred to as the intermediate region. In the implementation depicted in FIGS. 3 and 4, a three-piece construction of an absorbent article 210 is depicted where the absorbent article 210 can have a chassis 11 including a front waist panel 13 defining the front waist region 12, a rear waist panel 15 defining the rear waist region 14, and an absorbent panel 17 defining the crotch region 16 of the absorbent article 210. The absorbent panel 17 can extend between the front waist panel 13 and the rear waist panel 15. In some implementations, the absorbent panel 17 can overlap the front waist panel 13 and the rear waist panel 15. The absorbent panel 17 can be bonded to the front waist panel 13 and the rear waist panel 15 to define a three-piece construction. However, it is contemplated that an absorbent article can be manufactured in a cross-machine direction without being a three-piece construction garment.

The absorbent article 10, 210 can have a pair of longitudinal side edges 18, 20, and a pair of opposite waist edges, respectively designated front waist edge 22 and rear waist edge 24. In certain implementations, the front waist edge can include one or more moisture sensitive composites. In some implementations the rear waste edge can include one or more moisture sensitive composites. The front waist region 12 can be contiguous with the front waist edge 22 and the rear waist region 14 can be contiguous with the rear waist edge 24. The longitudinal side edges 18, 20 can extend from the front waist edge 22 to the rear waist edge 24. In certain implementations, the longitudinal side edges can include one or more moisture sensitive composites. The longitudinal side edges 18, 20 can extend in a direction parallel to the longitudinal direction 30 for their entire length, such as for the absorbent article 10 illustrated in FIGS. 1 and 2. In other implementations, the longitudinal side edges 18, 20 can be curved between the front waist edge 22 and the rear waist edge 24. In the absorbent article 210 of FIGS. 3 and 4, the longitudinal side edges 18, 20 can include portions of the front waist panel 13, the absorbent panel 17, and the rear waist panel 15.

The front waist region 12 can include the portion of the absorbent article 10, 210 that, when worn, is positioned at least in part on the front of the wearer while the rear waist region 14 can include the portion of the absorbent article 10, 210 that, when worn, is positioned at least in part on the back of the wearer. The crotch region 16 of the absorbent article 10, 210 can include the portion of the absorbent article 10, 210 that, when worn, is positioned between the legs of the wearer and can partially cover the lower torso of the wearer. The waist edges, 22 and 24, of the absorbent article 10, 210 are configured to encircle the waist of the wearer and together define a central waist opening 23 (as labeled in FIG. 1 and FIG. 3) for the waist of the wearer. Portions of the longitudinal side edges 18, 20 in the crotch region 16 can generally define leg openings for the legs of the wearer when the absorbent article 10, 210 is worn.

The absorbent article 10, 210 can include an outer cover 26 and a bodyside liner 28. The outer cover 26 and the bodyside liner 28 can form a portion of the chassis 11. In an implementation, the bodyside liner 28 can be bonded to the outer cover 26 in a superposed relation by any suitable means such as, but not limited to, adhesives, ultrasonic bonds, thermal bonds, pressure bonds, or other conventional techniques. The outer cover 26 can define a length in a longitudinal direction 30, and a width in the lateral direction 32, which, in the illustrated implementation, can coincide with the length and width of the absorbent article 10. As illustrated in FIGS. 2 and 4, the absorbent article 10, 210 can have a longitudinal axis 29 extending in the longitudinal direction 30 and a lateral axis 31 extending in the lateral direction 32.

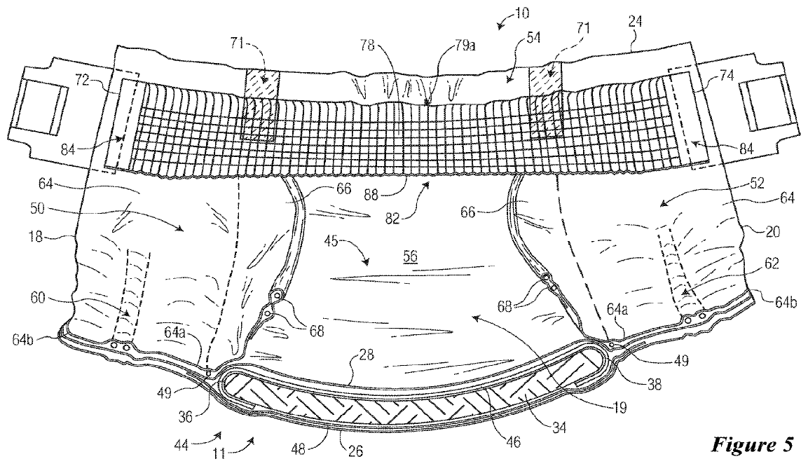

The chassis 11 can include an absorbent body 34. The absorbent body 34 can be disposed between the outer cover 26 and the bodyside liner 28. The absorbent body 34 can have longitudinal edges, 36 and 38, which, in an implementation, can form portions of the longitudinal side edges, 18 and 20, respectively, of the absorbent article 10, 210. The absorbent body 34 can have a first end edge 40 that is opposite a second end edge 42, respectively, which, in an implementation, can form portions of the waist edges, 22 and 24, respectively, of the absorbent article 10. In some implementations, the first end edge 40 can be in the front waist region 12. In some implementations, the second end edge 42 can be in the rear waist region 14. In an implementation, the absorbent body 34 can have a length and width that are the same as or less than the length and width of the absorbent article 10, 210. The bodyside liner 28, the outer cover 26, and the absorbent body 34 can form part of an absorbent assembly 44. In the absorbent article 210 of FIGS. 3 and 4, the absorbent panel 17 can form the absorbent assembly 44. The absorbent assembly 44 can also include a fluid transfer layer 46 (as shown in FIG. 5) and a fluid acquisition layer (not shown) between the bodyside liner 28 and the fluid transfer layer 46 as is known in the art. The absorbent assembly 44 can also include a spacer layer 48 (as shown in FIG. 5) disposed between the absorbent body 34 and the outer cover 26.

The absorbent article 10, 210 can be configured to contain and/or absorb liquid, solid, and semi-solid body exudates discharged from the wearer. In some implementations, containment flaps 50, 52 can be configured to provide a barrier to the lateral flow of body exudates. To further enhance containment and/or absorption of body exudates, the absorbent article 10, 210 can suitably include a waist containment member 54. In some implementations, the waist containment member 54 can be disposed in the rear waist region 14 of the absorbent article 10, 210. Although not depicted herein, it is contemplated that the waist containment member 54 can be additionally or alternatively disposed in the front waist region 12 of the absorbent article 10, 210.

The waist containment member 54 can be disposed on the body facing surface 19 of the chassis 11 to help contain and/or absorb body exudates. In some implementations, such as in the absorbent articles 10 depicted in FIGS. 1 and 2, the waist containment member 54 can be disposed on the body facing surface 45 of the absorbent assembly 44. In some implementations, the waist containment member 54 can be disposed on the body facing surface 56 of the bodyside liner 28. In some implementations, such as in the absorbent article 210 depicted in FIGS. 3 and 4, the waist containment member 54 can be disposed on the body facing surface 58 of the rear waist panel 15.

The absorbent article 10, 210 can further include leg elastic members 60, 62 as are known to those skilled in the art. In some implementations the leg elastic members include one or more moisture sensitive composites. The leg elastic members 60, 62 can be attached to the outer cover 26 and/or the bodyside liner 28 along the opposite longitudinal side edges, 18 and 20, and positioned in the crotch region 16 of the absorbent article 10, 210. The leg elastic members 60, 62 can be parallel to the longitudinal axis 29 as shown in FIGS. 2 and 4 or can be curved as is known in the art. The leg elastic members 60, 62 can be elastomeric and can provide elasticized leg cuffs.

In some implementations, the absorbent article 10, 210 can further include longitudinal extending fold lines 25a, 25b, as shown in FIGS. 2 and 4. The first longitudinal extending fold line 25a can be on one side of the longitudinal axis 29 of the absorbent article 10, 210 and the second longitudinal extending fold line 25b can be on an opposite side of the longitudinal axis 29. In some implementations, the longitudinal extending fold lines 25a, 25b can be generally parallel to the longitudinal axis 29 of the absorbent article 10, 210. In some implementations, the absorbent article 10, 210 can further include a lateral extending fold line 27. The lateral extending fold line 27 can be parallel to and located at the lateral axis 31 of the absorbent article 10, 210 in some implementations.

Additional details regarding each of these elements of the absorbent article 10, 210 described herein can be found below and with reference to the Figures.

### Outer Cover:

The outer cover 26 and/or portions thereof can be breathable and/or liquid impermeable. The outer cover 26 and/or portions thereof can be elastic, stretchable, or non-stretchable. The outer cover 26 may be constructed of a single layer, multiple layers, laminates, spunbond fabrics, films, meltblown fabrics, elastic netting, microporous webs, bonded-carded webs or foams provided by elastomeric or polymeric materials. In an implementation, for example, the outer cover 26 can be constructed of a microporous polymeric film, such as polyethylene or polypropylene.

In an implementation, the outer cover 26 can be a single layer of a liquid impermeable material, such as a polymeric film. In an implementation, the outer cover 26 can be suitably stretchable, and more suitably elastic, in at least the lateral direction 32 of the absorbent article 10, 210. In an implementation, the outer cover 26 can be stretchable, and more suitably elastic, in both the lateral 32 and the longitudinal 30 directions. In an implementation, the outer cover 26 can be a multi-layered laminate in which at least one of the layers is liquid impermeable. In some implementations, the outer cover 26 can be a two-layer construction, including an outer layer (not shown) and an inner layer (not shown) which can be bonded together such as by a laminate adhesive. Suitable laminate adhesives can be applied continuously or intermittently as beads, a spray, parallel swirls, or the like, but it is to be understood that the inner layer can be bonded to the outer layer by other bonding methods, including, but not limited to, ultrasonic bonds, thermal bonds, pressure bonds, or the like.

The outer layer of the outer cover 26 can be any suitable material and may be one that provides a generally cloth-like texture or appearance to the wearer. An example of such material can be a 100% polypropylene bonded-carded web with a diamond bond pattern available from Sandler A. G., Germany, such as 30 gsm Sawabond 4185® or equivalent. Another example of material suitable for use as an outer layer of an outer cover 26 can be a 20 gsm spunbond polypropylene non-woven web. The outer layer may also be constructed of the same materials from which the bodyside liner 28 can be constructed as described herein.

The liquid impermeable inner layer of the outer cover 26 (or the liquid impermeable outer cover 26 where the outer cover 26 is of a single-layer construction) can be either vapor permeable (i.e., “breathable”) or vapor impermeable. The liquid impermeable inner layer (or the liquid impermeable outer cover 26 where the outer cover 26 is of a single-layer construction) can be manufactured from a thin plastic film. The liquid impermeable inner layer (or the liquid impermeable outer cover 26 where the outer cover 26 is of a single-layer construction) can inhibit liquid body exudates from leaking out of the absorbent article 10, 210 and wetting articles, such as bed sheets and clothing, as well as the wearer and caregiver.

In some implementations, where the outer cover 26 is of a single layer construction, it can be embossed and/or matte finished to provide a more cloth-like texture or appearance. The outer cover 26 can permit vapors to escape from the absorbent article 10 while preventing liquids from passing through. A suitable liquid impermeable, vapor permeable material can be composed of a microporous polymer film or a non-woven material which has been coated or otherwise treated to impart a desired level of liquid impermeability.

The bodyside liner 28 of the absorbent article 10, 110, 210 can overlay the absorbent body 34 and the outer cover 26 and can isolate the wearer's skin from liquid waste retained by the absorbent body 34. In various implementations, a fluid transfer layer 46 can be positioned between the bodyside liner 28 and the absorbent body 34. In various implementations, an acquisition layer (not shown) can be positioned between the bodyside liner 28 and the absorbent body 34 or a fluid transfer layer 46, if present. In various implementations, the bodyside liner 28 can be bonded to the acquisition layer, or to the fluid transfer layer 46 if no acquisition layer is present, via adhesive and/or by a point fusion bonding. The point fusion bonding may be selected from ultrasonic, thermal, pressure bonding, and combinations thereof.

In an implementation, the bodyside liner 28 can extend beyond the absorbent body 34 and/or a fluid transfer layer 46, if present, and/or an acquisition layer, if present, and/or a spacer layer 48, if present, to overlay a portion of the outer cover 26 and can be bonded thereto by any method deemed suitable, such as, for example, by being bonded thereto by adhesive, to substantially enclose the absorbent body 34 between the outer cover 26 and the bodyside liner 28. The bodyside liner 28 may be narrower than the outer cover 26. However, in other implementations, the bodyside liner 28 and the outer cover 26 may be of the same dimensions in width and length. In other implementations, the bodyside liner 28 can be of greater width than the outer cover 26. It is also contemplated that the bodyside liner 28 may not extend beyond the absorbent body 34 and/or may not be secured to the outer cover 26. In some implementations, the bodyside liner 28 can wrap at least a portion of the absorbent body 34, including wrapping around both longitudinal edges 36, 38 of the absorbent body 34, and/or one or more of the end edges 40, 42. It is further contemplated that the bodyside liner 28 may be composed of more than one segment of material. The bodyside liner 28 can be of different shapes, including rectangular, hourglass, or any other shape. The bodyside liner 28 can be suitably compliant, soft feeling, and non-irritating to the wearer's skin and can be the same as or less hydrophilic than the absorbent body 34 to permit body exudates to readily penetrate through to the absorbent body 34 and provide a relatively dry surface to the wearer.

The bodyside liner 28 can be manufactured from a wide selection of materials, such as synthetic fibers (for example, polyester or polypropylene fibers), natural fibers (for example, wood or cotton fibers), a combination of natural and synthetic fibers, porous foams, reticulated foams, apertured plastic films, or the like. Examples of suitable materials include, but are not limited to, rayon, wood, cotton, polyester, polypropylene, polyethylene, nylon, or other heat-bondable fibers, polyolefins, such as, but not limited to, copolymers of polypropylene and polyethylene, linear low-density polyethylene, and aliphatic esters such as polylactic acid, finely perforated film webs, net materials, and the like, as well as combinations thereof.

Various woven and non-woven fabrics can be used for the bodyside liner 28. The bodyside liner 28 can include a woven fabric, a nonwoven fabric, a polymer film, a film-fabric laminate or the like, as well as combinations thereof. Examples of a nonwoven fabric can include spunbond fabric, meltblown fabric, coform fabric, carded web, bonded-carded web, bicomponent spunbond fabric, spunlace, or the like, as well as combinations thereof. The bodyside liner 28 need not be a unitary layer structure, and thus, can include more than one layer of fabrics, films, and/or webs, as well as combinations thereof. For example, the bodyside liner 28 can include a support layer and a projection layer that can be hydroentagled. The projection layer can include hollow projections, such as those disclosed in U.S. Pat. No. 9,474,660 to Kirby, Scott S. C. et al.

For example, the bodyside liner 28 can be composed of a meltblown or spunbond web of polyolefin fibers. Alternatively, the bodyside liner 28 can be a bonded-carded web composed of natural and/or synthetic fibers. The bodyside liner 28 can be composed of a substantially hydrophobic material, and the hydrophobic material can, optionally, be treated with a surfactant or otherwise processed to impart a desired level of wettability and hydrophilicity. The surfactant can be applied by any conventional means, such as spraying, printing, brush coating or the like. The surfactant can be applied to the entire bodyside liner 28, or it can be selectively applied to particular sections of the bodyside liner 28.

In an implementation, a bodyside liner 28 can be constructed of a non-woven bicomponent web. The non-woven bicomponent web can be a spunbonded bicomponent web, or a bonded-carded bicomponent web. An example of a bicomponent staple fiber includes a polyethylene/polypropylene bicomponent fiber. In this particular bicomponent fiber, the polypropylene forms the core and the polyethylene form the sheath of the fiber. Fibers having other orientations, such as multi-lobe, side-by-side, end-to-end may be used without departing from the scope of this disclosure. In an implementation, a bodyside liner 28 can be a spunbond substrate with a basis weight from 10 or 12 to 15 or 20 gsm. In an implementation, a bodyside liner 28 can be a 12 gsm spunbond-meltblown-spunbond substrate having 10% meltblown content applied between the two spunbond layers.

Although the outer cover 26 and bodyside liner 28 can include elastomeric materials, it is contemplated that the outer cover 26 and the bodyside liner 28 can be composed of materials which are generally non-elastomeric. In an implementation, the bodyside liner 28 can be stretchable, and more suitably elastic. In an implementation, the bodyside liner 28 can be suitably stretchable and more suitably elastic in at least the lateral or circumferential direction of the absorbent article 10, 210. In other aspects, the bodyside liner 28 can be stretchable, and more suitably elastic, in both the lateral and the longitudinal directions 32, 30, respectively.

### Containment Flaps:

In an implementation, the absorbent article 10, 210 can include a pair of containment flaps 50, 52. The containment flaps 50, 52 can be formed separately from the absorbent chassis 11 and attached to the chassis 11 or can be formed integral to the chassis 11. In some implementations, the containment flaps 50, 52 can be secured to the chassis 11 of the absorbent article 10, 210 in a generally parallel, spaced relation with each other laterally inward of the leg openings to provide a barrier against the flow of body exudates. One containment flap 50 can be on a first side of the longitudinal axis 29 and the other containment flap 52 can be on a second side of the longitudinal axis 29. In an implementation, the containment flaps 50, 52 can extend generally in a longitudinal direction 30 from the front waist region 12 of the absorbent article 10, through the crotch region 16 to the rear waist region 14 of the absorbent article 10. In some implementations, the containment flaps 50, 52 can extend in a direction substantially parallel to the longitudinal axis 29 of the absorbent article 10, 210, however, in other implementations, the containment flaps 50, 52 can be curved, as is known in the art. In other implementations, such as the absorbent article 210 in FIGS. 3 and 4, the containment flaps 50, 52 can be disposed on the absorbent panel 17 in the crotch region 16.

In implementations where the containment flaps 50, 52 are coupled to the chassis 11, the containment flaps 50, 52 can be bonded to the bodyside liner 28 with a barrier adhesive 49, as shown in FIG. 5. Alternatively, the containment flaps 50, 52 can be bonded to the outer cover 26 with a barrier adhesive 49, or to the spacer layer 48. Of course, the containment flaps 50, 52 can be bonded to other components of the chassis 11 and can be bonded with other suitable means other than a barrier adhesive 49. The containment flaps 50, 52 can be constructed of a fibrous material which can be similar to the material forming the bodyside liner 28. Other conventional materials, such as polymer films, can also be employed.

The containment flaps 50, 52 can each include a base portion 64 and a projection portion 66. The base portion 64 can be bonded to the chassis 11, for example, to the bodyside liner 28 or the outer cover 26 as mentioned above. The base portion 64 can include a proximal end 64a and a distal end 64b. The projection portion 66 can be separated from the base portion 64 at the proximal end 64a of the base portion 64. As used in this context, the projection portion 66 is separated from the base portion 64 at the proximal end 64a of the base portion 64 in that the proximal end 64a of the base portion 64 defines a transition between the projection portion 66 and the base portion 64. The proximal end 64a of the base portion 64 can be located near the barrier adhesive 49. In some implementations, the distal ends 64b of the base portion 64 can laterally extend to the respective longitudinal side edges 18, 20 of the absorbent article 10, 210. In other implementations, the distal ends 64b of the base portion 64 can end laterally inward of the respective longitudinal side edges 18, 20 of the absorbent article 10, 210. The containment flaps 50, 52 can also each include a projection portion 66 that is configured to extend away from the body facing surface 19 of the chassis 11 at least in the crotch region 16 when the absorbent article 10, 210 is in a relaxed configuration, as illustrated in FIG. 5. The containment flaps 50, 52 can include a tack-down region 71 in either or both of the front waist region 12 and the rear waist region 14 where the projection portion 66 is coupled to the body facing surface 19 of the chassis 11.

It is contemplated that the containment flaps 50, 52 can be of various configurations and shapes, and can be constructed by various methods. For example, the containment flaps 50, 52 of FIG. 5 depict a vertical containment flap 50, 52 with a tack-down region 71 in both the front and rear waist regions 12, 14 where the projection portion 66 of each containment flap 50, 52 is tacked down to the bodyside liner 28 towards or away from the longitudinal axis 29 of the absorbent article 10, 210. However, the containment flaps 50, 52 can include a tack-down region 71 where the projection portion 66 of each of the containment flaps 50, 52 is folded back upon itself and coupled to itself and the bodyside liner 28 in a “C-shape” configuration, as is known in the art and described in U.S. Pat. No. 5,895,382 to Robert L. Popp et al. As yet another alternative, it is contemplated that the containment flaps 50, 52 could be constructed in a “T-shape” configuration, such as described in U.S. Pat. No. 9,259,362 by Robert L. Popp et al. Such a configuration can also include a tack-down region 71 in either or both of the front and rear waist regions 12, 14, respectively. Of course, other configurations of containment flaps 50, 52 can be used in the absorbent article 10, 210 and still remain within the scope of this disclosure.

The containment flaps 50, 52 can include one or more flap elastic members 68, such as the two flap elastic strands depicted in FIG. 5. Suitable elastomeric materials for the flap elastic members 68 can include sheets, strands or ribbons of natural rubber, synthetic rubber, or thermoplastic elastomeric materials. Of course, while two elastic members 68 are shown in each containment flap 50, 52, it is contemplated that the containment flaps 50, 52 can be configured with one or three or more elastic members 68. Alternatively or additionally, the containment flaps 50, 52 can be composed of a material exhibiting elastomeric properties itself.

The flap elastic members 68, as illustrated in FIG. 5, can have two strands of elastomeric material extending longitudinally in the projection portion 66 of the containment flaps 50, 52, in generally parallel, spaced relation with each other. The elastic members 68 can be within the containment flaps 50, 52 while in an elastically contractible condition such that contraction of the strands gathers and shortens the projection portions 66 of the containment flaps 50, 52 in the longitudinal direction 30. As a result, the elastic members 68 can bias the projection portions 66 of the containment flaps 50, 52 to extend away from the body facing surface 45 of the absorbent assembly 44 in a generally upright orientation of the containment flaps 50, 52, especially in the crotch region 16 of the absorbent article 10, 210, when the absorbent article 10 is in a relaxed configuration.

During manufacture of the containment flaps 50, 52 at least a portion of the elastic members 68 can be bonded to the containment flaps 50, 52 while the elastic members 68 are elongated. The percent elongation of the elastic members 68 can be, for example, 110% to 350%. In one implementation, the elastic members 68 can be coated with adhesive while elongated to a specified length prior to attaching to the elastic members 68 to the containment flaps 50, 52. In a stretched condition, the length of the clastic members 68 which have adhesive coupled thereto can provide an active flap elastic region 70 in the containment flaps 50, 52, as labeled in FIG. 2, which will gather upon relaxation of the absorbent article 10. The active flap elastic region 70 of containment flaps 50, 52 can be of a longitudinal length that is less than the length of the absorbent article 10, 210. In this example method of bonding the elastic members 68 to the containment flaps 50, 52, the portion of the elastic members 68 not coated with adhesive, will retract after the elastic members 68 and the absorbent article 10 are cut in manufacturing to form an individual absorbent article 10. As noted above, the relaxing of the elastic members 68 in the active flap elastic region 70 when the absorbent article 10, 210 is in a relaxed condition can cause each containment flap 50, 52 to gather and cause the projection portion 66 of each containment flap 50, 52 to extend away from the body facing surface 19 of the chassis 11 (e.g., the body facing surface 45 of the absorbent assembly 44 or the body facing surface 56 of the bodyside liner 28), as depicted in FIG. 5.

Of course, the elastic members 68 can be bonded to the containment flaps 50, 52 in various other ways as known by those of skill in the art to provide an active flap elastic region 70, which is within the scope of this disclosure. Additionally, the active flap elastic regions 70 can be shorter or longer than depicted herein, including extending to the front waist edge 22 and the rear waist edge 24, and still be within the scope of this disclosure.

### Leg Elastics:

Leg elastic members 60, 62 can be secured to the outer cover 26, such as by being bonded thereto by laminate adhesive, generally laterally inward of the longitudinal side edges, 18 and 20, of the absorbent article 10, 210. The leg elastic members 60, 62 can form elasticized leg cuffs that further help to contain body exudates. In an implementation, the leg elastic members 60, 62 may be disposed between inner and outer layers (not shown) of the outer cover 26 or between other layers of the absorbent article 10, for example, between the base portion 64 of each containment flap 50, 52 and the bodyside liner 28 as depicted in FIG. 5, between the base portion 64 of each containment flap 50, 52 and the outer cover 26, or between the bodyside liner 28 and the outer cover 26. The leg elastic members 60, 62 can be one or more elastic components near each longitudinal side edge 18, 20. For example, the leg elastic members 60, 62 as illustrated herein each include two elastic strands. A wide variety of elastomeric materials may be used for the leg clastic members 60, 62.

Suitable elastomeric materials can include sheets, strands or ribbons of natural rubber, synthetic rubber, or thermoplastic elastomeric materials. The elastomeric materials can be stretched and secured to a substrate, secured to a gathered substrate, or secured to a substrate and then elasticized or shrunk, for example, with the application of heat, such that the elastic retractive forces are imparted to the substrate. Additionally, it is contemplated that the leg elastic members 60, 62 can be formed with the containment flaps 50, 52, and then attached to the chassis 11 in some implementations. Of course, the leg elastic members 60, 62 can be omitted from the absorbent article 10, 210 without departing from the scope of this disclosure.

### Waist Containment Member:

In an implementation, the absorbent article 10, 210 can have one or more waist containment members 54. The waist containment member(s) 54 can be disposed in the rear waist region 14 as illustrated in FIGS. 1-5. In general, the waist containment member 54 can help contain and/or absorb body exudates, especially low viscosity fecal matter, and as such, can be preferred to be in the rear waist region 14. In some implementations, the absorbent article 10, 210 can have a waist containment member 54 disposed in the front waist region 12. A waist containment member 54 in the front waist region 12 can help contain and/or absorb body exudates, such as urine, in the front waist region 12. Although not as prevalent as in the rear waist region 14, in some circumstances, fecal material may also spread to the front waist region 12, and thus, a waist containment member 54 disposed in the front waist region 12 can help contain and/or absorb body exudates as well. In other implementations, the absorbent article 10, 210 can have a waist containment member 54 in both the rear waist region 14 and the front waist region 12.

The waist containment member 54 can be disposed on the body facing surface 45 of the absorbent assembly 44. In some implementations, such as in implementations illustrated in FIGS. 1-2 and 5, the waist containment member 54 can be disposed on the body facing surface 56 of the bodyside liner 28. However, in some implementations, such as the absorbent article 210 in FIG. 4, the waist containment member 54 can be disposed on a body facing surface 58 of the rear waist panel 15.

The waist containment member 54 can include a first longitudinal side edge 72 and a second longitudinal side edge 74. The first longitudinal side edge 72 can be opposite from the second longitudinal side edge 74. The distance between the first longitudinal side edge 72 and the second longitudinal side edge 74 can define a width 51 of the waist containment member 54 in the lateral direction 32, as shown in FIG. 2.

As illustrated in FIGS. 2 and 5, the waist containment member 54 can be configured such that the first longitudinal side edge 72 can be disposed laterally outward of the proximal end 64a of the base portion 64 of the containment flap 50. Similarly, the waist containment member 54 can be configured such that the second longitudinal side edge 74 can be disposed laterally outward of the proximal end 64a of the base portion 64 of the containment flap 52. The waist containment member 54 can be configured such that the width 51 of the waist containment member 54 can be greater than a lateral distance between longitudinal extending fold lines 25a, 25b, as shown in FIGS. 2 and 4.

The waist containment member 54 can also include a proximal portion (not shown) and a distal portion 78. The proximal portion can be coupled to the body facing surface 19 of chassis 11 (e.g., the body facing surface 45 of the absorbent assembly 44 or the body facing surface 56 of the bodyside liner 28) whereas the distal portion 78 of the waist containment member 54 can be free to move with respect to the chassis 11 and the absorbent assembly 44 when the absorbent article 10, 210 is in the relaxed configuration, such as shown in FIG. 5. When the waist containment member 54 is in a relaxed configuration, the distal portion 78 extends away from the chassis 11 and absorbent assembly 44 in a vertical direction, which is perpendicular to the plane defined by the longitudinal axis 29 and the lateral axis 31. A fold 79a can separate the proximal portion from the distal portion 78 of the waist containment member 54. As used in this context, the fold 79a separates the proximal portion from the distal portion 78 in that the fold 79a defines a transition between the proximal portion and the distal portion 78.

In some implementations, the proximal portion of the waist containment member 54 can be coupled to the body facing surface 56 of the bodyside liner 28. In other implementations, the proximal portion of the waist containment member 54 can be coupled to the body facing surface 58 of the rear waist panel 15. The proximal portion can be coupled to the body facing surface 45 by an adhesive, by pressure bonding, by ultrasonic bonding, by thermal bonding, and combinations thereof.

Because the distal portion 78 of the waist containment member 54 can freely move with respect to the absorbent assembly 44 when the absorbent article 10, 210 is in the relaxed configuration, the distal portion 78 can help provide a containment pocket 82 when the absorbent article 10, 210 is in the relaxed configuration. The containment pocket 82 can help provide a barrier to contain and/or can help absorb body exudates. The containment pocket 82 can be especially beneficial for containing and/or absorbing low viscosity fecal matter, which can be prevalent in younger children. The first longitudinal side edge 72 can be disposed laterally outward of the proximal end 64a of the base portion 64 of the containment flap 50, and thus, the containment pocket 82 can extend laterally outward of the proximal end 64a of the containment flap 50. Similarly, the second longitudinal side edge 74 can be disposed laterally outward of the proximal end 64a of the base portion 64 of the containment flap 52 and the containment pocket 82 can extend laterally outward of the proximal end 64a of the containment flap 52. Such a configuration provides waist containment member 54 with a wide containment pocket 82 to contain and/or absorb body exudates.

To help prevent lateral flow of body exudates that are contained by the containment pocket 82 of the waist containment member 54, the distal portion 78 of the waist containment member 54 can be bonded to the proximal portion of the waist containment member 54 and/or the body facing surface 19 of the chassis 11 near the first and second longitudinal side edges 72, 74, respectively. For example, FIG. 5 depicts tack-down regions 84 where the distal portion 78 of the waist containment member 54 can be bonded to the proximal portion of the waist containment member 54 and/or the body facing surface 19 of the chassis 11.

In preferred implementations, the waist containment member 54 can include at least one elastic member and even more elastic members in further implementations. Generally, the elastic member can span substantially from the first longitudinal side edge 72 to the second longitudinal side edge 74 of the waist containment member 54. The elastic member can be disposed in the distal portion 78 of the waist containment member 54, and preferably, is located near a free edge 88 of the distal portion 78 of the waist containment member 54.

A wide variety of elastomeric materials may be used for the elastic member(s) in the waist containment member 54. Suitable elastomeric materials can include sheets, strands or ribbons of natural rubber, synthetic rubber, elastic foams, or thermoplastic elastomeric materials (e.g., films). The elastomeric materials can be stretched and secured to a substrate forming the waist containment member 54, secured to a gathered substrate, or secured to a substrate and then elasticized or shrunk, for example, with the application of heat, such that the clastic retractive forces are imparted to the substrate forming the waist containment member 54.

The waist containment member 54 can be disposed to be coupled to the chassis 11 by being placed either over the containment flaps 50, 52 or under the containment flaps 50, 52. More specifically, the waist containment member 54 can be disposed on the body facing surface 19 of the chassis 11 such that the proximal portion of the waist containment member 54 is disposed over the base portion 64 of the first and the second containment flaps 50, 52, respectively. Alternatively, the waist containment member 54 can be disposed on the body facing surface 19 of the chassis 11 such that the proximal portion of the waist containment member 54 is disposed under the base portion 64 of the first and the second containment flaps 50, 52, respectively. Both configurations can provide advantages to the functioning of the waist containment member 54 to contain and/or absorb body exudates.

Where the proximal portion of the waist containment member 54 is disposed over the base portion 64 of the containment flaps 50, 52, the containment flaps 50, 52 can have an active flap elastic region 70 that longitudinally overlaps with the distal portion 78 of the waist containment member 54 when the absorbent article 10 is in the stretched, laid flat configuration, such as illustrated in FIG. 2. Additionally or alternatively, the tack-down region 71 may not extend from the rear waist edge 24 to the free edge 88 of the distal portion 78 of the waist containment member 54, such as illustrated in FIG. 2.

Where the proximal portion of the waist containment member 54 is disposed under the base portion 64 of the containment flaps 50, 52, the tack-down region 71 of the projection portion 66 of each of the containment flaps 50, 52 may longitudinally overlap with the distal portion 78 of the waist containment member 54. In some of these implementations, the tack-down region 71 of projection portion 66 of each of the containment flaps 50, 52 can extend to the free edge 88 of the waist containment member 54 to further assist in containing exudates to the containment pocket 82 created by the waist containment member 54.

The waist containment member 54 can be comprised of a variety of materials. In a preferred implementation, the waist containment member 54 can be comprised of a spunbond-meltblown-spunbond (“SMS”) material. However, it is contemplated that the waist containment member 54 can be comprised of other materials including, but not limited to, a spunbond-film-spunbond (“SFS”), a bonded carded web (“BOW”), or any non-woven material. In some implementations, the waist containment member 54 can be comprised of a laminate of more than one of these example materials, or other materials. In some implementations, the waist containment member 54 can be comprised of a liquid impermeable material. In some implementations, the waist containment member 54 can be comprised of a material coated with a hydrophobic coating. The basis weight of the material forming the waist containment member 54 can vary, however, in a preferred implementation, the basis weight can be between 8 gsm to 120 gsm, not including the elastic members 86 in the waist containment member 54. More preferably, the basis weight of the material comprising the waist containment member 54 can be between 10 gsm to 40 gsm, and even more preferably, between 15 gsm to 25 gsm.

In various implementations, the absorbent article 10 can include a fastening system, wherein the fastening system includes the polymeric cohesive attachment according to the present disclosure. The fastening system can include one or more back fasteners 91 and one or more front fasteners 92. The implementations shown in FIGS. 1 and 2 depict implementations with one front fastener 92. Portions of the fastening system may be included in the front waist region 12, rear waist region 14, or both.

The fastening system can be configured to secure the absorbent article 10 the waist of the wearer in a fastened condition as shown in FIG. 1 and help maintain the absorbent article 10 in place during use. In an implementation, the back fasteners 91 can include one or more moisture sensitive composites according to the present disclosure. materials bonded together to form a composite ear as is known in the art. For example, the composite fastener may be composed of a stretch component 94, a nonwoven carrier or hook base 96, and a polymeric cohesive attachment component 98, as labeled in FIG. 2. As shown in FIG. 5, in some implementations the waist containment member 54 can extend to back fasteners 91. In some implementations, the waist containment member 54 can be coupled to the stretch component 94 of the back fasteners 91, either directly or indirectly. In some implementations, the waist containment member 54 can extend to the longitudinal side edges 18, 20 of the absorbent article 10, 210.

### Absorbent Body:

The absorbent body 34 can be suitably constructed to be generally compressible, conformable, pliable, non-irritating to the wearer's skin and capable of absorbing and retaining liquid body exudates. The absorbent body 34 can be manufactured in a wide variety of sizes and shapes (for example, rectangular, trapezoidal, T-shape, l-shape, hourglass shape, etc.) and from a wide variety of materials. The size and the absorbent capacity of the absorbent body 34 should be compatible with the size of the intended wearer (infants to adults) and the liquid loading imparted by the intended use of the absorbent article 10, 210. The absorbent body 34 can have a length and width that can be less than or equal to the length and width of the absorbent article 10, 210.

In an implementation, the absorbent body 34 can be composed of absorbent material, such as fibrous absorbent material and/or, superabsorbent material, binder materials, surfactants, selected hydrophobic and hydrophilic materials, pigments, lotions, odor control agents or the like, as well as combinations thereof. In an implementation, the absorbent body 34 can be a matrix of cellulosic fluff and superabsorbent material. In another implementation, the absorbent material of the absorbent body 34 can comprise only superabsorbent material. In an implementation, the absorbent body 34 may be constructed of a single layer of materials, or in the alternative, may be constructed of two or more layers of materials.

When composed at least partially of fibrous material, various types of wettable, hydrophilic fibers can be used in the absorbent body 34. Examples of suitable fibers include natural fibers, cellulosic fibers, synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber.

When composed at least partially of superabsorbent materials, such superabsorbent materials can be selected from natural, synthetic, and modified natural polymers and materials. The superabsorbent materials can be inorganic materials, such as silica gels, or organic compounds, such as cross-linked polymers.

If a spacer layer 48 is present, the absorbent body 34 can be disposed on the spacer layer 48 and superposed over the outer cover 26. The spacer layer 48 can be bonded to the outer cover 26, for example, by adhesive. In some implementations, a spacer layer 48 may not be present and the absorbent body 34 can directly contact the outer cover 26 and can be directly bonded to the outer cover 26. However, it is to be understood that the absorbent body 34 may be in contact with, and not bonded with, the outer cover 26 and remain within the scope of this disclosure. In an implementation, the outer cover 26 can be composed of a single layer and the absorbent body 34 can be in contact with the singer layer of the outer cover 26. In some implementations, at least a portion of a layer, such as but not limited to, a fluid transfer layer 46 and/or a spacer layer 48, can be positioned between the absorbent body 34 and the outer cover 26, such as illustrated in FIG. 5. The absorbent body 34 can be bonded to the fluid transfer layer 46 and/or the spacer layer 48.

Although the FIGS. 1-5 focus on description of a diaper absorbent article 10, 210, it should be understood that the moisture sensitive composites of the present disclosure may be used in any absorbent article-including but not limited to diapers, diaper pants, training pants, youth pants, swim pants, feminine hygiene products, including, but not limited to, menstrual pads or pants, incontinence products and other adult care garments, medical garments, surgical pads and bandages, other personal care or health care garments, and the like.

### EXAMPLES

The following examples are for the purpose of illustration of the invention only and are not intended to limit the scope of the present invention in any manner whatsoever.

Unless specified to the contrary, the polyethylene oxide (PEO) used for the following experiments was Polyox WSR-N80, MW_{w}=200,000; Dow.

### Preparation and Evaluation of Example Moisture-Responsive Polymer Systems

Various moisture-responsive polymer systems were prepared and experimentally tested by DSC. Polyethylene oxide (“PEO”) (was combined with Kraton G1645 (“K”), Vistamaxx 6102FL (Exxon Chem.) (“V”) in the weight ratios listed below and extruded into films.

TABLE 1

| | Heat (J/mg) | | T melting (° C.) | | Related degree |
|---|---|---|---|---|---|
| Comp. | (2^{nd} heating) | | (2^{nd} heating) | | of crystallinity |
| 100% PEO | −179 | (−133.9) | 68.4 | (63.1) | 1.00 |
| 100% V | −16.2 | (−21.6) | 55.4 | (35.7) | 0 |
| 45% PEO, 55% V | −66.6 | (−55.4) | 64.9 | (62.9) | 0.37 |
| 57% PEO, 43% V | −79.5 | (−74.2) | 65.5 | (62.8) | 0.44 |
| 65% PEO, 35% V | −91.1 | (−90.0) | 65.6 | (63.6) | 0.51 |
| 41% PEO, 49% K/V (3/7) | −56.7 | (−51.5) | 64.2 | (65.6) | 0.32 |
| 61.5% PEO, 38.5% K/V (3/7) | −81.3 | (−77.8) | 63.3 | (62.7) | 0.45 |
| 68% PEO, 32% K/V (3/7) | −91.3 | (−90.1) | 64.2 | (66.4) | 0.51 |

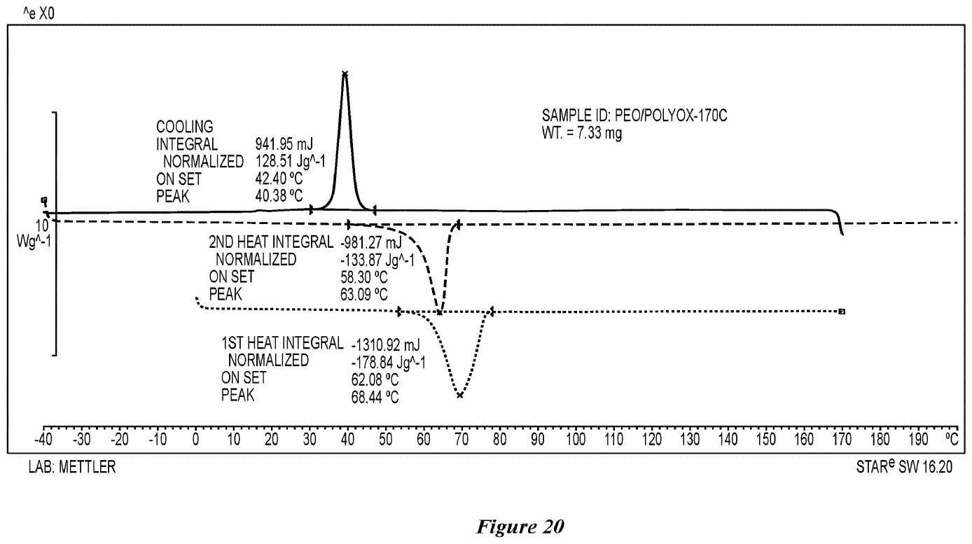

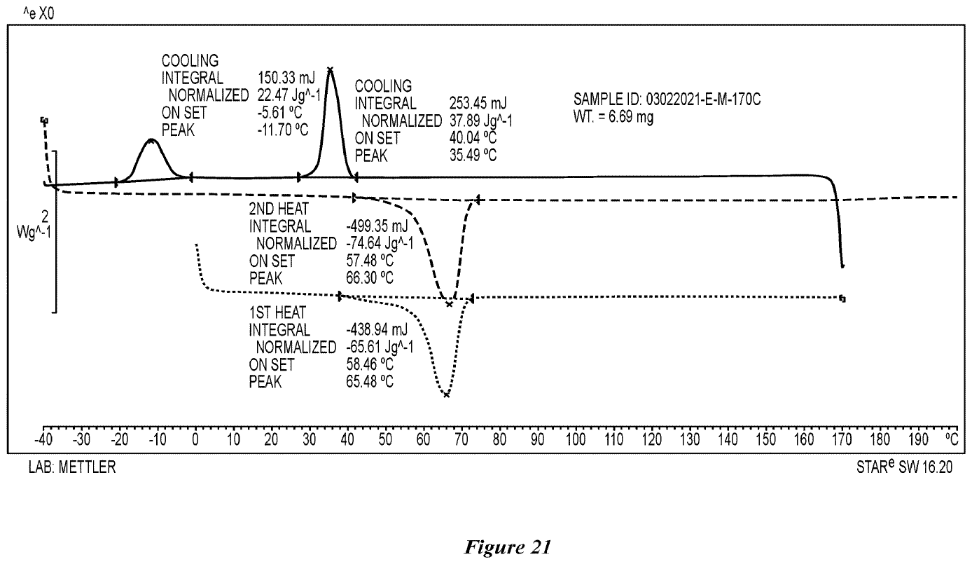

The melting point of crystalline polyethylene oxide (“PEO”) is around 65-68° C. As shown in Table 1, the degree of crystalline PEO is proportional to the PEO percentage in the blends. FIG. 20 depicts a DSC of 100% PEO. FIG. 21 depicts a DSC of 40% K/V=1:1/60% PEO. However, as PEO is not very compatible to elastomer chemically, the PEO degree of crystalline PEO is less related to the elastomer composition (as is also reflected in Table 1). Increasing PEO content resulted in increased plasticity, decreased elastic recovery, and made the film more difficult to stretch.

In addition, a mixture containing 60 wt. % PEO and 40 wt. % of a Kraton G1645 (Styrene-ethylene-propylene-styrene block copolymer)/Vistamaxx 6102FL (polyolefin elastomer from Exxon Chem.) mixture (1:1 ratio) was coextruded to form a film. FIG. 4 depicts the multicycle elastic behavior of this film. The stress vs. strain curve at 150% showed around 1000 g force. When this blend was combined with PEO, the material becomes completely pseudo plastic when stretched to 300%. After one week in room conditions, 80% of the stretched length remains. The length is reduced to 40% when immersed in saline for 1 minute. The elastic performance of the activated pseudo plastic is close to the original 100% elastomer.

Various prepared films were tested for tensile properties (peak stress, modulus, strain at break, and energy per volume at break). These measurements were performed using a strip elongation test which is substantially in accordance with the specifications in ASTM D5459-95. Specifically, the test uses two clamps each having two jaws with each jaw having a facing in contact with the sample. The clamps hold the material in the same plane usually vertically, separated by 1 inch and move the cross head at a specific rate of extension. The film samples were die cut in 2 inch wide by 6 inch long (in machine direction) portions, at a cross-head displacement rate of 20 in/min. The specimens are clamped in an MTS (Mechanical Test Systems) electromechanical test frame which has data acquisition capability. The tests were conducted under ambient conditions. Results are reported in the Figures herein as an average of at least five specimens.

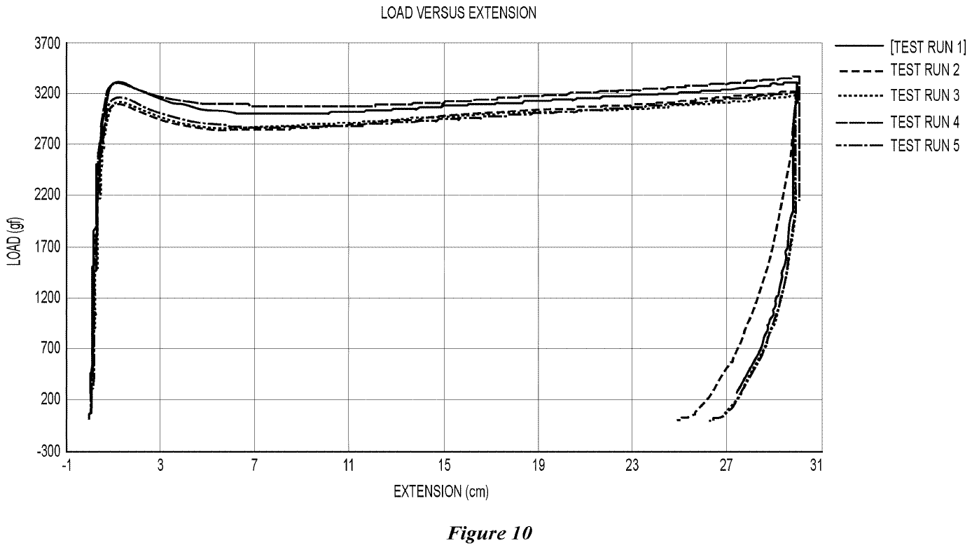

FIG. 10 depicts the load for stretching to 300% the original length of a 60 wt. % PEO, 40% Kraton G1645/Vistamaxx 6102 (1:1 ratio) film.

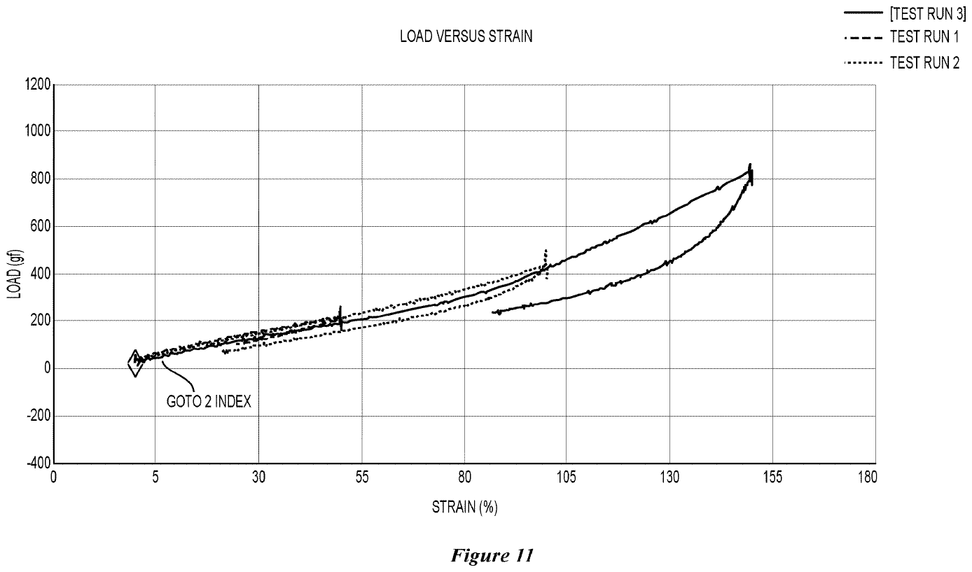

The elastic tension of the pseudo plastic can be modified by using a styrene-butadiene-styrene Kraton D polymer and polyolefin Vistamaxx with PEO blend. FIG. 11 depicts the elastic tension of 100% Kraton D1161 with around 500 g force at 300% strain.

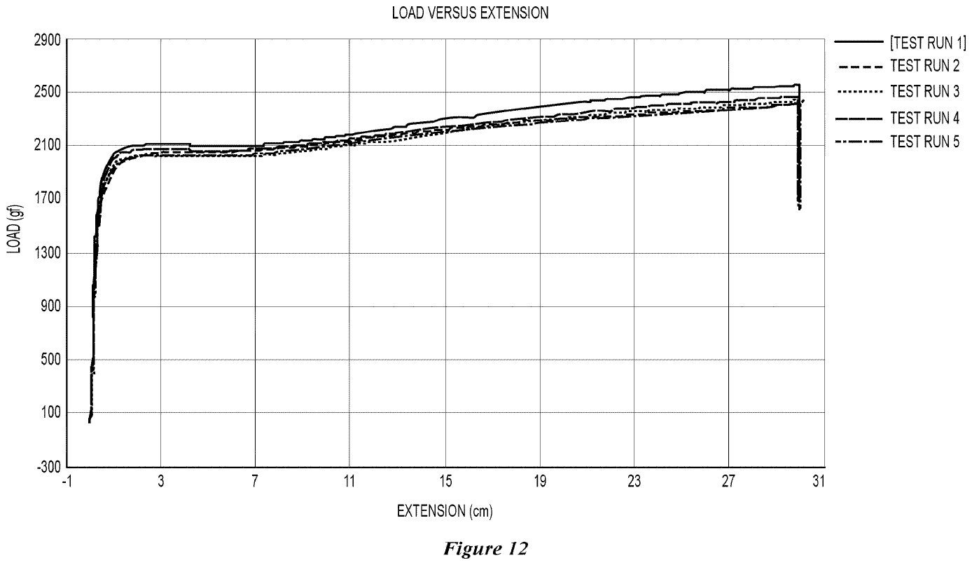

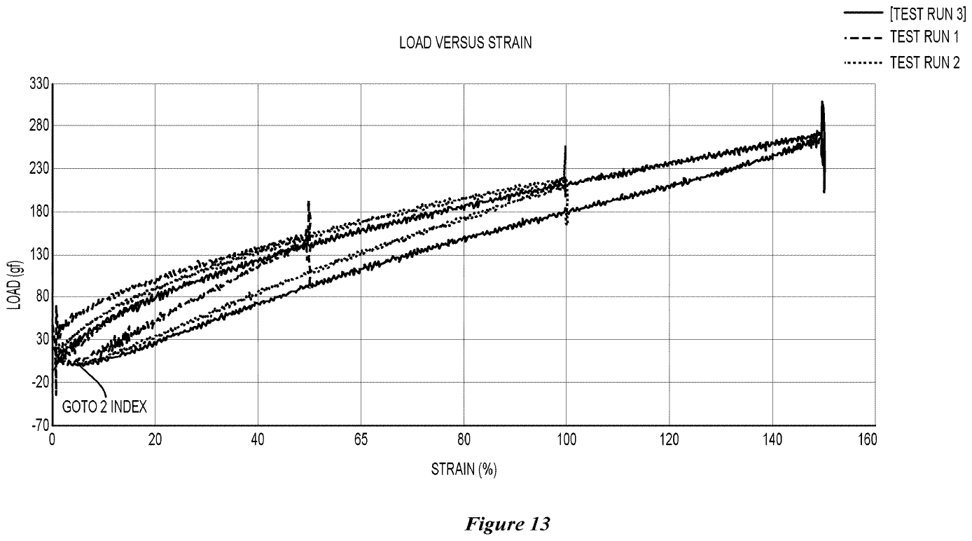

FIG. 12 depicts the pseudo elasticity of a 60 wt. % PEO, 40 wt. % D1161 moisture-responsive polymer system, indicating a stretching force around 2200 g. After the film is activated by contact with saline the activated elastic tension is about 300 g at 150% stretching (FIG. 13).

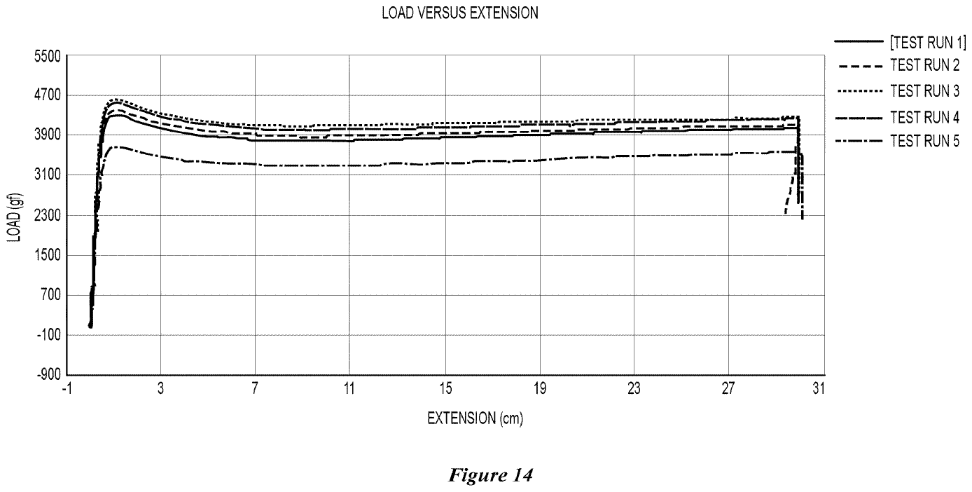

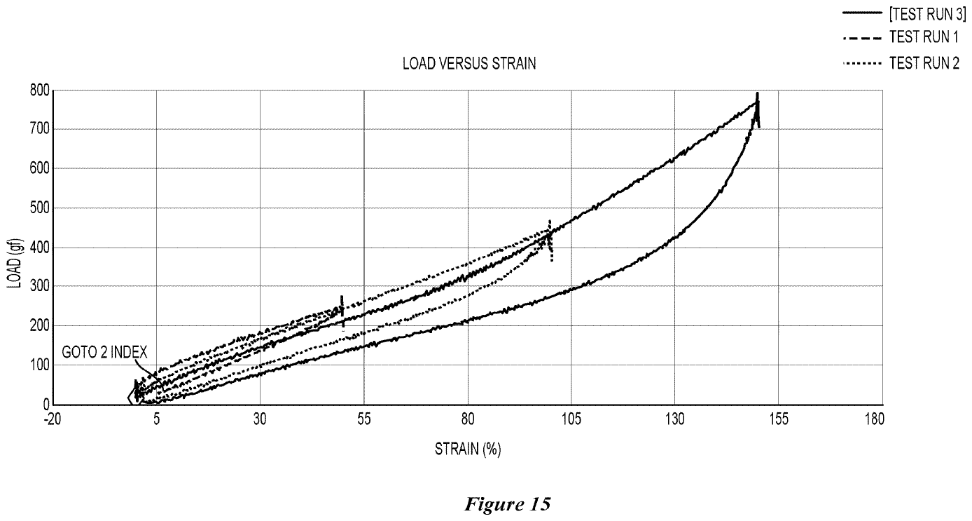

The addition of a second elastomer (Vistamaxx) increases the elastic tension dramatically for the blend of D1161+ Vistamaxx/PEO. FIG. 14 depicts stretching to stop at 300% for the film with 60 wt. % PEO, 40 wt. % (D1161/Vistamaxx=1:1), while FIG. 15 depicts multicycle testing after activation in saline for the 60 wt. % PEO, 40 wt. % (D1161/Vistamaxx=1:1). The stretching force for the pseudo elastic blend is around 4500 g (FIG. 14) and the elastic tension up to 800 g after activation with saline (FIG. 15).

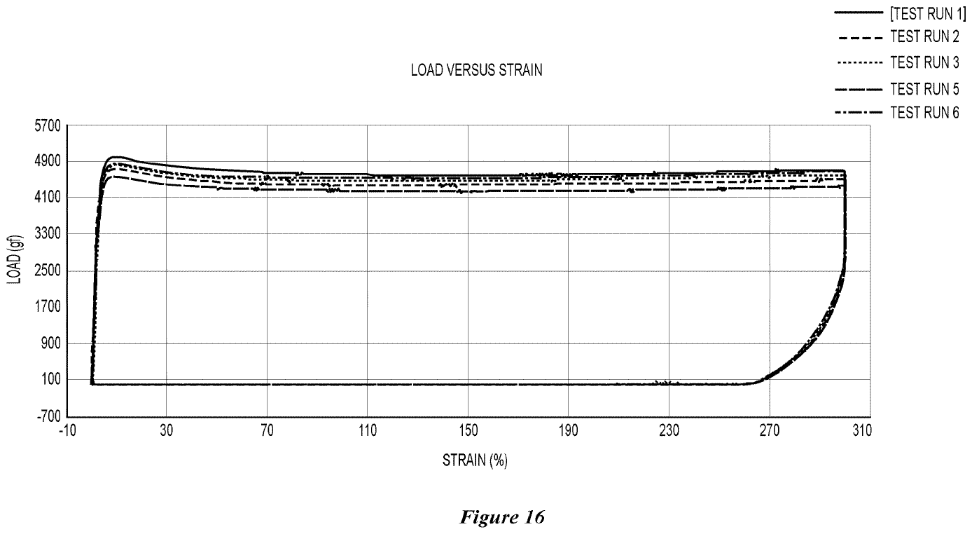

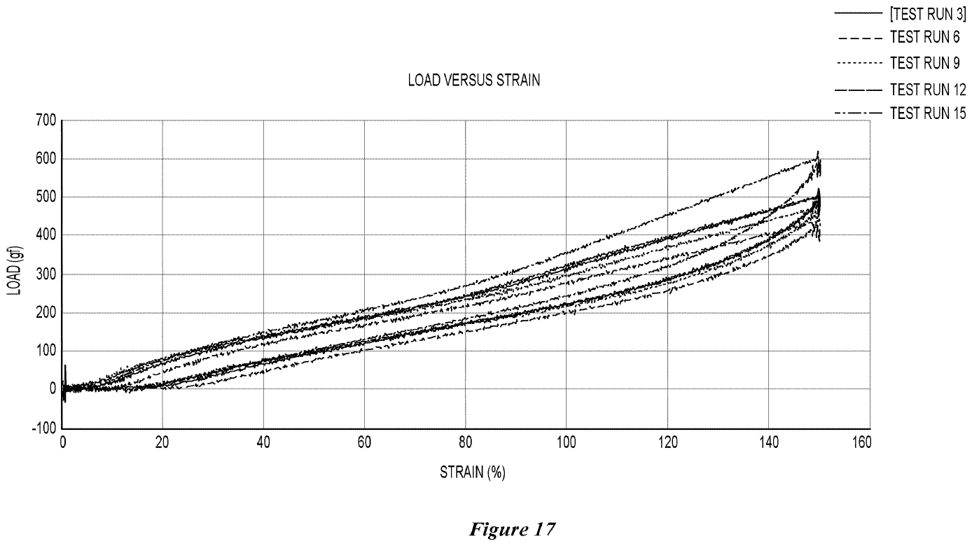

FIG. 16 depicts stretching to stop at 300% for a film containing 60 wt. % PEO, 40 wt. % (80 wt. % D1161, 15 wt. % CaCO₃, 5 wt. % PE (Dowlox 2407)), while FIG. 12 depicts elastic testing after activation in saline for the film containing 60 wt. % PEO, 40 wt. % (80 wt. % D1161, 15 wt. % CaCO3, 5 wt. % PE (Dowlox 2407)). The addition of CaCO₃ into pseudo plastic blend of D1161/PEO resulted in much higher (double) tension recovery after activation (FIG. 16) as well stretching force up to 4900 g (FIG. 17). The CaCO₃ particles did not improve elasticity but increased modulus and wet strength of the blend after activation.

As shown in Table 2, the shrinkage rate in saline was evaluated for multiple moisture-responsive polymer systems containing one or more of Vistamaxx 6102 (“V”), Kraton G1645 (“G1645), or Kraton D1161 (“D1161”).

TABLE 2

| Composition | Remaining length | Shrinkage (1 min) |
|---|---|---|
| 60 wt. % PEO | 94% | None (10 min) |
| 40 wt. % V | | |
| 60 wt. % PEO | 86% | 40% |
| 40 wt. % G1645/V (1:1) 90 gsm | | |
| 60 wt. % PEO | 86% | >50% |
| 40 wt. % G1657/V (1:1) 70 gsm | | |
| 60 wt. % PEO | >90% | >80% |
| 40 wt. % D1161/V (1:1) 98 gsm | | |
| 50 wt. % PEO | 93% | >40% |
| 50 wt. % D1161 104 gsm | | |
| 60 wt. % PEO | 100% | >90% |
| 40 wt. % D1161, 104 gsm | | |

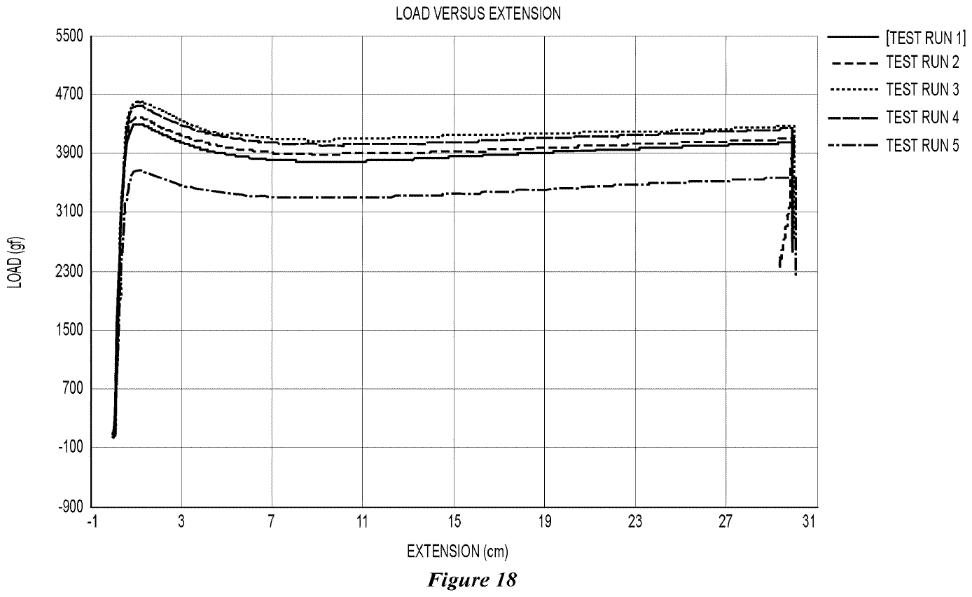

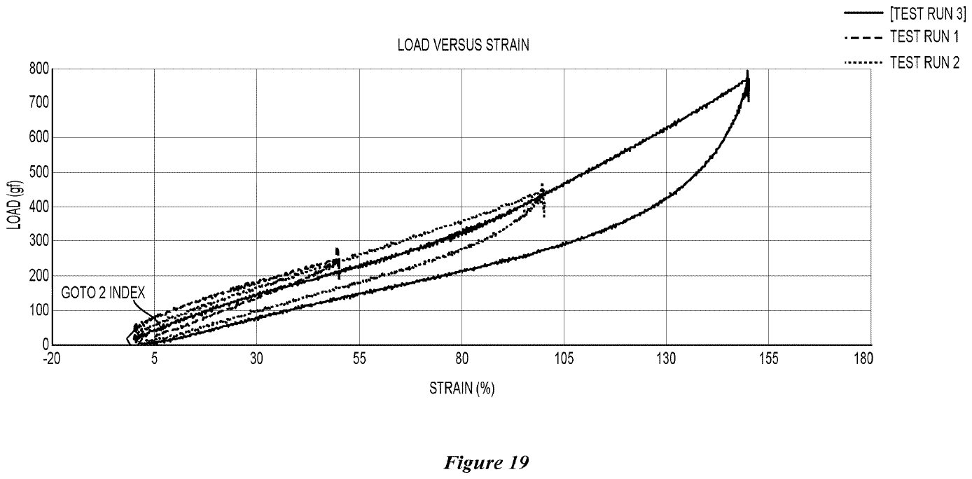

A 25 mm×25 mm film containing 60 wt. % PEO, 40 wt. % (Kraton D1161/Vistamaxx 6102 (1:1 ratio) was stretched to 300% its original length. The original width of the film was 25 mm before stretching, 90 gsm basis weight, and the width was reduced to 10 mm after stretching. The stretched film was stable in room temperature and ~50% RH, with no shrinkage observed after one week as shown in the FIG. 18. The pseudo elastic film was shrunk from 4 inches to about 1.5 inches after activation by immersing in water for 3 minutes. The activated film is elastic and the elastic film stress vs. strain test is shown in FIG. 19. The tensile testing of the activated elastic film indicated a peak load 1130 gf @ 300%.

As shown in Tables 3 and 4, the moisture-responsive polymer system was then bonded to various substrates: SB (12 gsm), SMS (42 gsm), BCW (100 gsm), LDPE Film (18 gsm), PE Film (26 gsm), PE Film (90 gsm), and PP Film (150 gsm). Double sided adhesive tape was used to bond these substrates to the pseudo elastic film. The pseudo laminate bending behavior was tested by dropping 1 mL of water on the surface of the moisture-responsive polymer system with a syringe. The laminate bending speed was measured by the bending angle vs time after exposure as shown below in Table 4.

TABLE 3

| | | | | LDPE | PE | PE | PP |
|---|---|---|---|---|---|---|---|
| Backing Material | SB | SMS | BCW | film | film | Film | Film |
| BWt (gsm) | 12 | 42 | 100 | 18 | 26 | 90 | 150 |
| Tensile peak load | 1582 | 3309 | 2800 | 444 | 790 | 3024 | 6510 |
| (gf) | | | | | | | |
| Bending | | x | x | | | x | x |
| Shrinking | x | | | x | x | | |

TABLE 4

| | |
|---|---|
| Backing Material | Bending (degrees) |

| (gsm) | 1 min | 3 min | 5 min | 7 min | 9 min |
|---|---|---|---|---|---|
| SMS, 42 | 5 | 20 | 55 | 180 | — |
| PE Film, 90 | 0 | 10 | 40 | 180 | — |
| SB, 12 | Shrinking | | | | |
| Poise* | 15 | 25 | 35 | 45 | 55 |
| BCW, 100 | 0 | <5 | 90 | 180 | — |
| PP film, 150 | 15 | 40 | 90 | 140 | 180 |

| | |
|---|---|
| PE film, 26 | Shrinking |
| PE Film, 41 | Shrinking, some bending |
| LDPE Film, 18 | Shrinking |

*with two strips of moisture-responsive polymer system laid side by side.
SMS = spunbond-meltblown-spunbond
SB = spunbond
Poise = POISE ® Pad (commercially available from Kimberly-Clark)
BCW = bonded-carded webs

### Preparation and Evaluation of Example Moisture-Responsive Composites

A moisture-responsive composite material can be made from laminating of a pseudo plastic film with a higher modulus thermoplastic/elastic sheet or paper using a polyolefin based hot melt adhesive (e.g., with add on 2-4 gsm). The laminates are not elastic, unable to be compressed at room temperature without permanent damage.

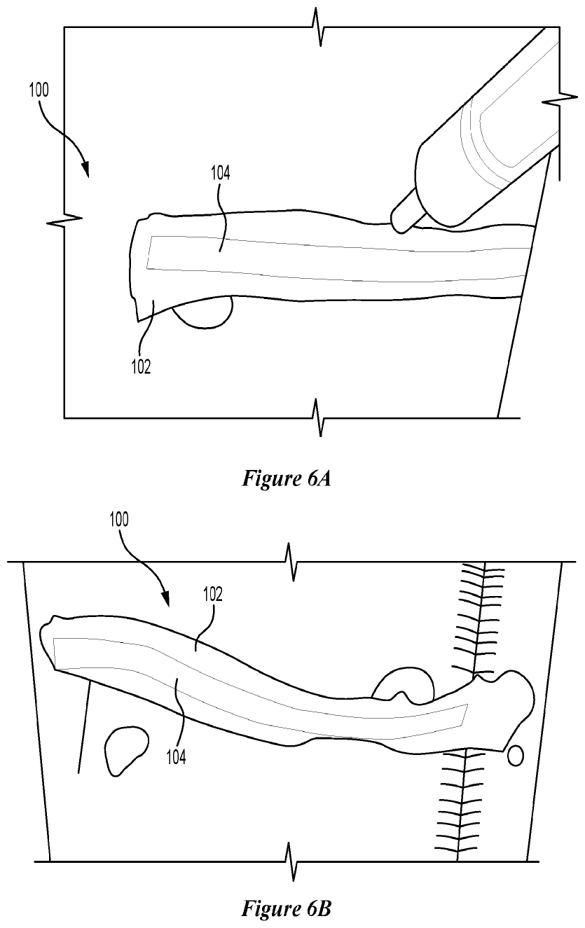

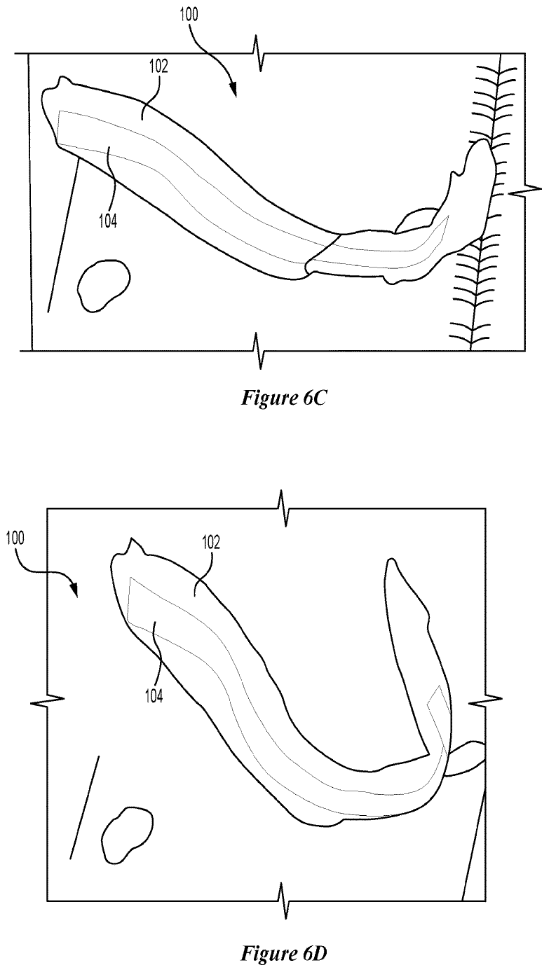

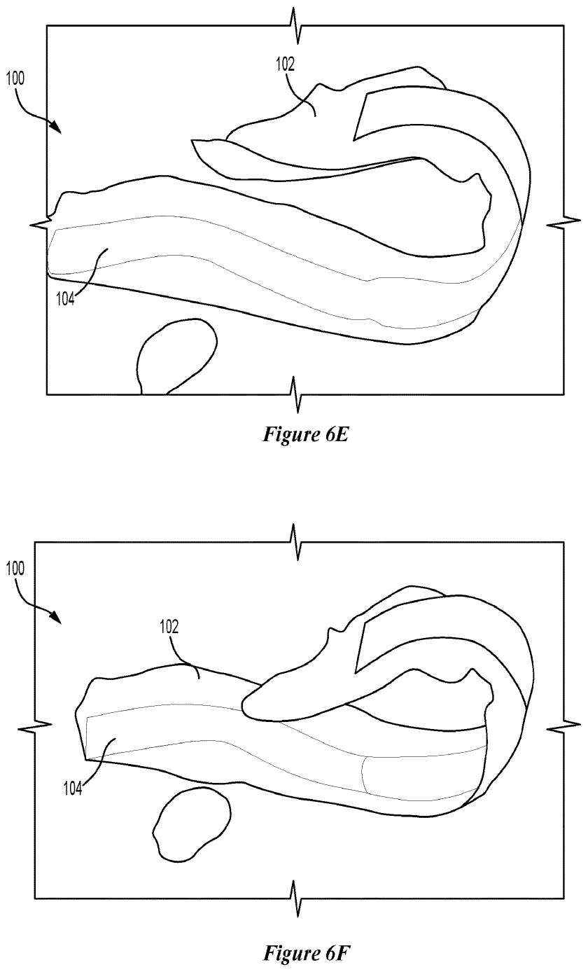

An exemplary moisture-responsive composite (100) is depicted in FIG. 6. A moisture-responsive film layer (102) was formed from a moisture-responsive polymer system comprising 60 wt. % PEO and 40 wt. % (D1161/Vistamaxx 6102 (1:1 ratio)). The moisture-responsive film layer (102) was then laminated to a coated paper (104) (100 gsm). FIGS. 6A-6F depict the action of moisture on the composite. Saline solution was dropped on the surface of the moisture-responsive polymer system (FIG. 6A) and bending commenced within 15 seconds. The remainder of FIG. 6 depicts the composite after 30 seconds (FIG. 6B), 60 seconds (FIG. 6C), 80 seconds (FIG. 6D), 100 seconds (FIG. 6E), and 120 seconds (FIG. 6F). The bending accelerated from 50-60 sec and completed bending in 2 min (c-f). Minimal amounts of saline were absorbed into the composite (estimated absorption <30% wt.).

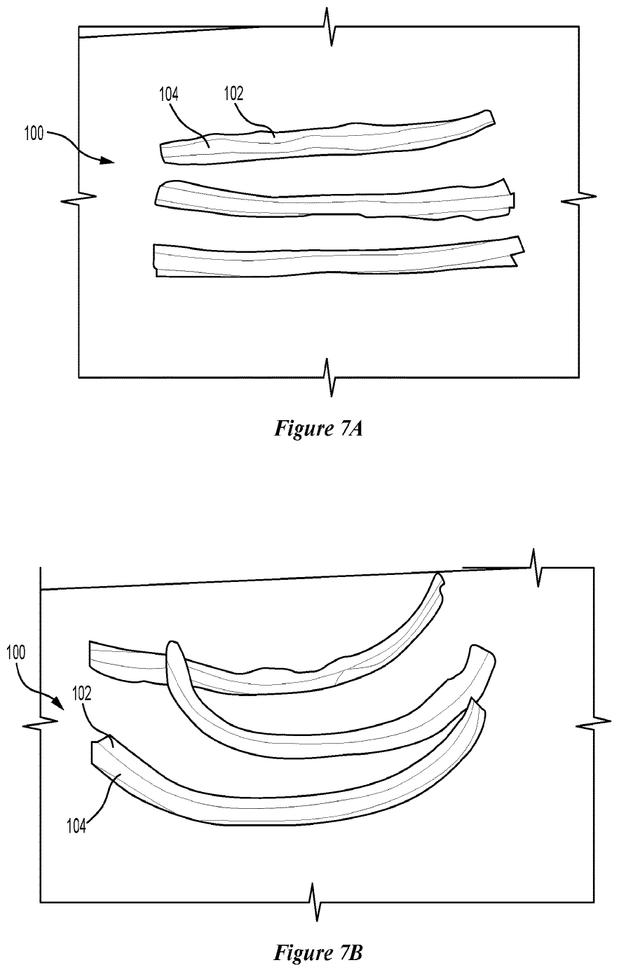

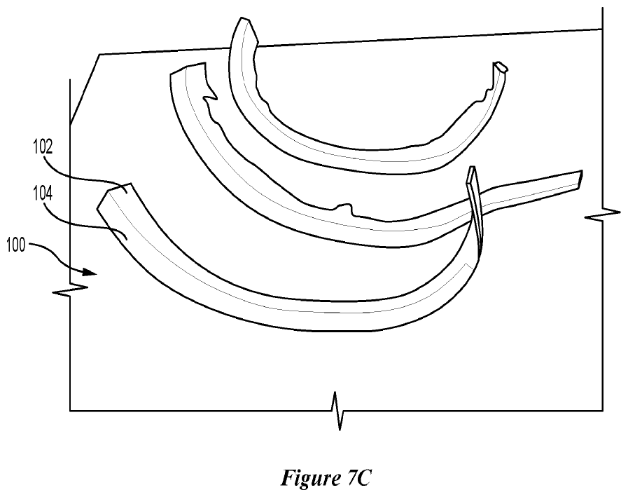

A pseudo plastic laminate (200) was constructed with (i) a moisture-responsive film layer (202) comprised of 60% PEO and 40% (Kraton D1161:Vistamaxx 6102=1:1), stretched 300%, with basis weight around 50 gsm, and (ii) a coated paper (204) adhesively laminated to the moisture-responsive film layer. The laminate was left on exposure to the atmosphere at room temperature with relative humidity of 50-60%. FIG. 7 depicts the movement of the composite at the time of creation (FIG. 7A), after two days (FIG. 7B), and after one week (FIG. 7C).

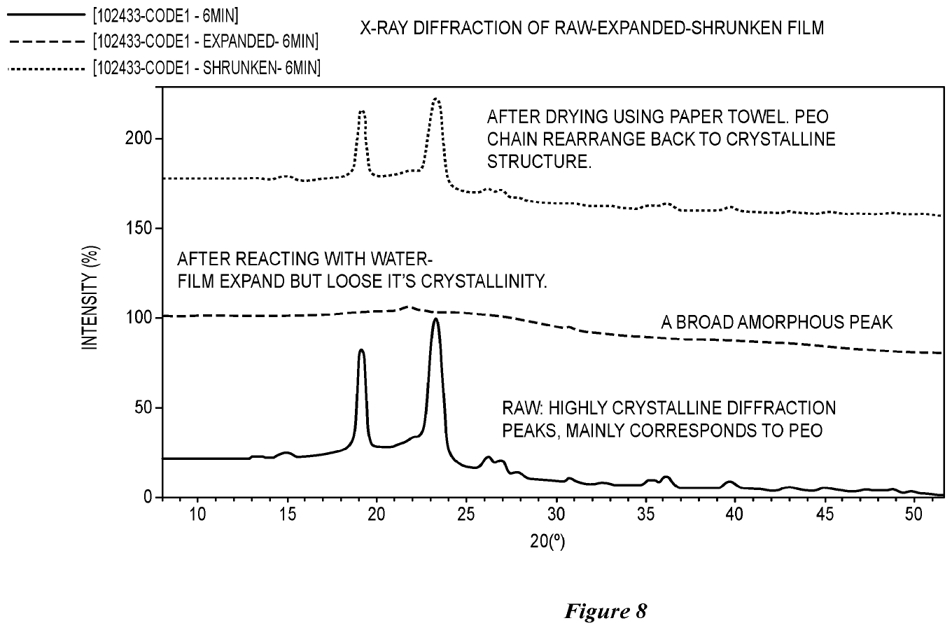

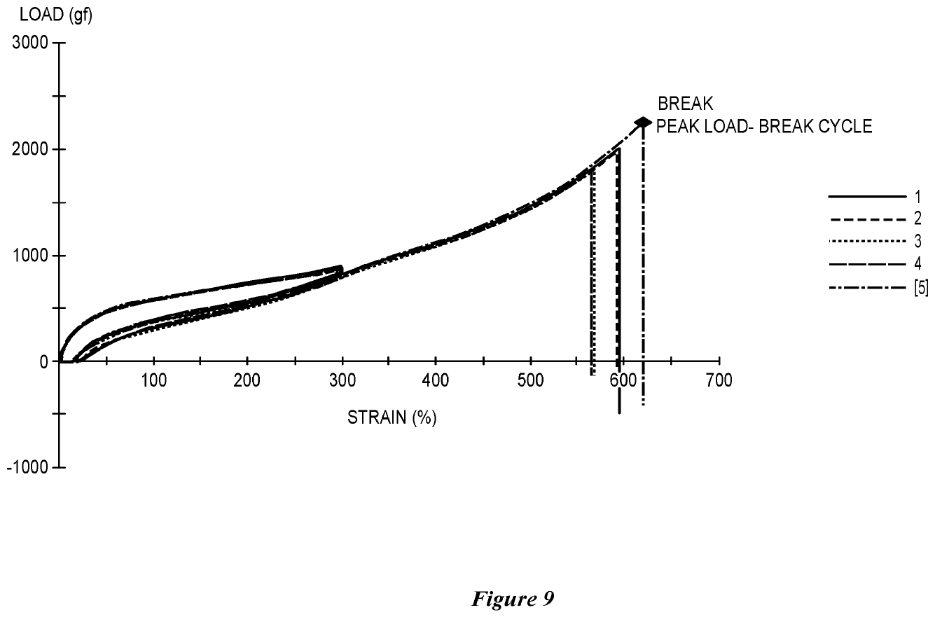

Without bounding by any theory, the movement is believed to be caused by release of elastic tension previously held by PEO crystals. The elastic tension is generated during the stretching of the polymer system, and this tension is held in place by crystalline PEO. Upon exposure to water, the crystalline lattice is disrupted thereby releasing the tension. The x-ray diffraction analysis depicted in FIG. 8 shows the initial crystalline form, the amorphous form resulting from contact with water, and the re-adoption of crystalline form following drying. The shrinkage of the film is restrained by the high modulus of the other layer so that a bending moment is created. The higher of the shrinkage force of the moisture-responsive material and higher modulus of the moisture-responsive backing layer create higher degree of bending.

### ADDITIONAL EMBODIMENTS

Example 1. A moisture-responsive composite comprising: a moisture-responsive layer comprising a moisture-responsive polymer system; and a backing layer laminated with the moisture-responsive layer; wherein, upon exposure to moisture, the moisture-responsive composite bends from a first position to a second position.

Example 2. The moisture-responsive composite according to any preceding Example, wherein the moisture-responsive layer is elongated and defines a longitudinal axis; and wherein the moisture-responsive composite's longitudinal axis defines a radius of curvature that decreases as the moisture-responsive composite bends from the first position to the second position.

Example 3. The moisture-responsive composite according to any preceding Example, wherein the moisture-responsive composite defines a first end and a second end in the first position, and wherein the first end and second end are closer together in the second position than in the first position.

Example 4. The moisture-responsive composite according to any preceding Example, wherein the movement from first position towards second position decreases the radius of curvature of the center line by at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

Example 5. The moisture-responsive composite according to any preceding Example, wherein the backing layer has a higher elastic modulus than the moisture-responsive layer.

Example 6. The moisture-responsive composite according to any preceding Example, wherein the elastic modulus of the backing layer is at least 110%, at least 125%, at least 150%, at least 200%, at least 250%, at least 300%, at least 400%, or at least 500% the elastic modulus of the moisture-responsive layer.

Example 7. The moisture-responsive composite according to any preceding Example, wherein the backing layer has a higher Young's modulus than the moisture-responsive layer.

Example 8. The moisture-responsive composite according to any preceding Example, wherein the Young's modulus of the backing layer is at least 110%, at least 125%, at least 150%, at least 200%, at least 250%, at least 300%, at least 400%, or at least 500% the Young's modulus of the moisture-responsive layer.

Example 9. The moisture-responsive composite according to any preceding Example, wherein the backing layer has a higher flexural modulus than the moisture-responsive layer.

Example 10. The moisture-responsive composite according to any preceding Example, wherein the flexural modulus of the backing layer is at least 110%, at least 125%, at least 150%, at least 200%, at least 250%, at least 300%, at least 400%, or at least 500% the flexural modulus of the moisture-responsive layer.

Example 11. The moisture-responsive composite according to any preceding Example, wherein the backing layer has a higher bending stiffness than the moisture-responsive layer.

Example 12. The moisture-responsive composite according to any preceding Example, wherein the bending stiffness of the backing layer is at least 110%, at least 125%, at least 150%, at least 200%, at least 250%, at least 300%, at least 400%, or at least 500% the bending stiffness of the moisture-responsive layer.

Example 13. The moisture-responsive composite according to any preceding Example, wherein the moisture-responsive system has a thickness from, from 0.01-1 mm, from 0.01-0.5 mm, from 0.01-0.25 mm, from 0.01-0.1 mm, from 0.05-1 mm, from 0.1-1 mm, from 0.5-1.5 mm, from 0.5-2.5 mm, or from 1-2.5 mm.

Example 14. The moisture-responsive composite according to any preceding Example, wherein the moisture-responsive polymer system has a basis weight between about 1-1,000, about 50-1,000, about 100-1,000, about 1-500, about 1-250, about 5-250, about 10-250, about 10-100, about 10-50, about 25-50, about 25-75, about 50-100, about 75-125, about 100-150, about 50-150, about 100-250, about 250-500, or about 500-1,000 grams per square meter.

Example 15. The moisture-responsive composite according to any preceding Example, wherein elongated moisture-responsive polymer system has a stretch ratio from 2-6, 2-4, 3-6, 3-6, 3-7, or 3-8.

Example 16. The moisture-responsive composite according to any preceding Example, wherein the elongated moisture-responsive polymer system is stretched to a length that is 2-6 times, 2-4 times, 3-6 times, 3-6 times, 3-7 times, or 3-8 times the length of the moisture-responsive polymer system prior to stretching.

Example 17. The moisture-responsive composite according to any preceding Example, wherein the elongated moisture-responsive polymer system has a basis weight (gsm) that is no more than 90%, no more than 80%, no more than 70%, no more than 60%, no more than 50%, no more than 40%, no more than 30%, or no more than 20% the basis weight of the moisture-responsive polymer system prior to stretching.

Example 18. The moisture-responsive composite according to any preceding Example, wherein the elongated moisture-responsive polymer system has a width that is no more than 90%, no more than 80%, no more than 70%, no more than 60%, no more than 50%, no more than 40%, no more than 30%, or no more than 20% the width of the moisture-responsive polymer system prior to stretching.

Example 19. The moisture-responsive composite according to any preceding Example, wherein the moisture-responsive polymer system comprises a hydrophilic polymer and an elastomer.

Example 20. The moisture-responsive composite according to any preceding Example, wherein the moisture-responsive polymer system comprises a hydrophilic polymer and an elastomer in a wt. ratio (hydrophilic polymer:elastomer) from: no more than 75 wt. % hydrophilic polymer, e.g., from 25:75 to 75:25, from 30:70 to 75:25, from 35:65 to 75:25, from 40:60 to 75:25, from 45:55 to 75:25, from 50:50 to 75:25, from 55:45 to 75:25, from 60:40 to 75:25, from 65:35 to 75:25; no more than 70 wt. % hydrophilic polymer, e.g., from 25:75 to 70:30, from 30:70 to 70:30, from 35:65 to 70:30, from 40:60 to 70:30, from 45:55 to 70:30, from 50:50 to 70:30, from 55:45 to 70:30, from 60:40 to 70:30, from 65:35 to 70:30; no more than 65 wt. % hydrophilic polymer, e.g., from 25:75 to 65:35, from 30:70 to 65:35, from 35:65 to 65:35, from 40:60 to 65:35, from 45:55 to 65:35, from 50:50 to 65:35, from 55:45 to 65:35, from 60:30 to 65:35; no more than 60 wt. % hydrophilic polymer, e.g., from 25:75 to 60:40, from 30:70 to 60:40, from 35:65 to 60:40, from 40:60 to 60:40, from 45:55 to 60:40, from 50:50 to 60:40, from 55:45 to 60:40; no more than 55 wt. % hydrophilic polymer, e.g., from 25:75 to 55:45, from 30:70 to 55:45, from 35:65 to 55:45, from 40:60 to 55:45, from 45:55 to 55:45, from 50:50 to 55:45; no more than 50 wt. % hydrophilic polymer, e.g., from 25:75 to 50:50, from 30:70 to 50:50, from 35:65 to 50:50, from 40:60 to 50:50, from 45:55 to 50:50; no more than 45 wt. % hydrophilic polymer, e.g., from 25:75 to 45:55, from 30:70 to 45:55, from 35:65 to 45:55, from 40:60 to 45:55; no more than 40 wt. % hydrophilic polymer, e.g., from 25:75 to 40:60, from 30:70 to 40:60, from 35:65 to 40:60; or no more than 35 wt. % hydrophilic polymer, e.g., from 25:75 to 35:65, from 30:70 to 35:65.

Example 21. The moisture-responsive composite according to any preceding Example, wherein the moisture-responsive polymer system comprises a hydrophilic polymer and an elastomer in a wt. ratio (hydrophilic polymer:elastomer) from 70:30 to 50:50.

Example 22. The moisture-responsive composite according to any preceding Example, wherein the moisture-responsive polymer system comprises a hydrophilic polymer and an elastomer in a wt. ratio (hydrophilic polymer:elastomer) from 70:30 to 55:45.

Example 23. The moisture-responsive composite according to any preceding Example, wherein the moisture-responsive polymer system comprises a hydrophilic polymer and an elastomer in a wt. ratio (hydrophilic polymer:elastomer) from 70:30 to 60:40.

Example 24. The moisture-responsive composite according to any preceding Example, wherein the moisture-responsive polymer system comprises a hydrophilic polymer and an elastomer in a wt. ratio (hydrophilic polymer:elastomer) from 60:40 to 50:50.

Example 25. The moisture-responsive composite according to any preceding Example, wherein the moisture-responsive polymer system comprises a hydrophilic polymer having a MW_{w} from 10,000-1,000,000 Da, from 10,000-500,000 Da, from 50,000-500,000 Da, from 50,000-250,000 Da, from 50,000-1000,000, from 100,000-500,000 Da, from 100,000-300,000 Da, from 200,000-400,000 Da, from 150,000-300,000 Da, or from 250,000-500,000 Da.

Example 26. The moisture-responsive composite according to any preceding Example, wherein the moisture-responsive polymer system comprises a poly(vinyl pyrrolidone), poly(hydroxyethyl (meth)acrylate, poly(hydroxypropyl (meth)acrylate, poly(meth)acrylic acid, poly(vinyl pyridine), poly(meth)acrylamide, poly(vinyl acetate), poly(vinyl alcohol), poly(ethylene oxide), or a combination thereof.

Example 27. The moisture-responsive composite according to any preceding Example, wherein the moisture-responsive polymer system comprises poly(ethylene oxide).

Example 28. The moisture-responsive composite according to any preceding Example, wherein the moisture-responsive polymer system comprises a poly(ethylene oxide) homopolymer, a poly(ethylene oxide-co-propylene oxide) block copolymer, or a combination thereof.

Example 29. The moisture-responsive composite according to any preceding Example, wherein the hydrophilic polymer has a solubility in water, at 23° C., of at least 0.1 g/mL, at least 0.5 g/mL, at least 1 g/mL, at least 2.5 g/mL, at least 5 g/mL, or at least 10 g/mL.

Example 30. The moisture-responsive composite according to any preceding Example, wherein when the moisture-responsive composite is in first position, the hydrophilic polymer has a degree of crystallinity of at least 25%, at least 30%, at least 40%, at least 50%, at least 60%, at least 70%, at least 80%, or at least 90%.

Example 31. The moisture-responsive composite according to any preceding Example, wherein when the moisture-responsive composite is in first position, the hydrophilic polymer has a degree of crystallinity of at least 25%, at least 30%, at least 40%, at least 50%, at least 60%, at least 70%, at least 80%, or at least 90%, and when submerged in water, converts to at least 90% amorphous form within 120 seconds, 100 seconds, 80 seconds, 60 seconds, 50 seconds, 40 seconds, 30 seconds, 20 seconds, or at least 10 seconds.

Example 32. The moisture-responsive composite according to any preceding Example, wherein the moisture-responsive polymer system comprises an elastomer having a MW_{w} from 1,000-1,000,000 Da, from 1,000-10,000 Da, from 1,000-5,000 Da, from 2,500-10,000 Da, from 2,500-7,500 Da, from 2,500-10,000 Da, from 5,000-10,000 Da, from 5,000-25,000 Da, from 10,000-20,000 Da, from 10,000-50,000 Da, from 10,000-100,000, from 10,000-250,000 Da, from 25,000-250,000 Da, from 50,000-250,000 Da, from 50,000-100,000 Da, from 100,000-250,000 Da, from 10,000-500,000 Da, from 50,000-500,000 Da, from 100,000-500,000 Da, from 100,000-300,000 Da, from 200,000-400,000 Da, from 150,000-300,000 Da, or from 250,000-500,000 Da.

Example 33. The moisture-responsive composite according to any preceding Example, wherein the elastomer comprises a polyolefin elastomer.

Example 34. The moisture-responsive composite according to any preceding Example, wherein the elastomer comprises a styrene block copolymer, a butadiene block copolymer, a hydrogenated butadiene block copolymer, an isoprene block copolymer, a hydrogenated isoprene block copolymer, non-crystalline ethylene/α-olefin random copolymer, low-crystalline ethylene/α-olefin random copolymer, propylene/ethylene/α-olefin random copolymer, or combination thereof.

Example 35. The moisture-responsive composite according to any preceding Example, wherein the elastomer comprises polystyrene-polybutadiene-polystyrene block copolymer (SBS), polystyrene-polyisoprene-polystyrene block copolymer (SIS), polystyrene-poly/ethylene/butylene-polystyrene block copolymer (SEBS), polystyrene-poly/ethylene/propylene-polystyrene block copolymer, or a combination thereof.

Example 36. The moisture-responsive composite according to any preceding Example, wherein the elastomer comprises a polyolefin elastomer comprising an ethylene/propylene random copolymer, ethylene/1-butene random copolymer, propylene/1-butene random copolymer, or a combination thereof.

Example 37. The moisture-responsive composite according to any preceding Example, wherein the elastomer comprises a polyester elastomer, polyamide elastomer, or a combination thereof.

Example 38. The moisture-responsive composite according to any preceding Example, wherein the elastomer comprises an ethylene/α-olefin copolymer, propylene/α-olefin copolymer, styrene-olefin copolymer, or a combination thereof.

Example 39. The moisture-responsive composite according to any preceding Example, wherein the elastomer comprises poly(ethylene-butene), poly(ethylene-hexene), poly(ethylene-octene), poly(ethylene-propylene), poly(styrene-butadiene-styrene), poly(styrene-isoprene-styrene), poly(styrene-ethylene-butylene-styrene), poly(ester-ether), poly(ether-amide), poly(ethylene-vinylacetate), poly(ethylene-methylacrylate), poly(ethylene-acrylic acid), poly(ethylene butylacrylate), polyurethane, poly(ethylene-propylene-diene), ethylene-propylene rubber, or a combination thereof.

Example 40. The moisture-responsive composite according to any preceding Example, wherein the backing layer comprises a hydrophobic material, a hydrophilic material, or a combination thereof.

Example 41. The moisture-responsive composite according to any preceding Example, wherein the backing layer comprises nonwoven materials, films, wetlaid, airlaid, spunbond, meltblown, coform, bonded-carded webs, foams, tissue, netting, scrim, woven materials, or a combination thereof.

Example 42. The moisture-responsive composite according to any preceding Example, wherein the backing layer has a basis weight between about 1-1,000, grams per square meter, for example 1-50, 1-25, 1-10, 1-5, 5-10, 5-15, 10-20, 10-25, 10-50, or 25-50 grams per square meter.

Example 43. The moisture-responsive composite according to any preceding Example, wherein the backing layer has a basis weight between about 1-1,000, grams per square meter, for example about 1-1,000, about 50-1,000, about 100-1,000, about 1-500, about 1-250, about 5-250, about 10-250, about 10-100, about 50-100, about 75-125, about 100-150, about 50-150, about 100-250, about 250-500, or about 500-1,000 grams per square meter.

Example 44. The moisture-responsive composite according to any preceding Example, wherein the moisture-responsive layer is adhesively laminated or autogenously laminated to the backing layer.

Example 45. The moisture-responsive composite according to any preceding Example, wherein the moisture-responsive layer is laminated to the backing layer using ultrasonic bonding, thermal bonding, pressure bonding through-air bonding, calendar bonding, or a combination thereof.

Example 46. The moisture-responsive composite according to any preceding Example, wherein the moisture-responsive layer is adhesively laminated to the backing layer by a hot melt adhesive, a pressure-sensitive adhesive, or a combination thereof.

Example 47. The moisture-responsive composite according to any preceding Example, further comprising an adhesion layer disposed between the backing layer and the moisture-responsive layer.

Example 48. The moisture-responsive composite according to any preceding Example, wherein the adhesion layer has a basis weight between about 1-10, 1-5, 1-3, 2-5, 3-7, 5-10, 5-25, or 10-25 grams per square meter.

Example 49. The moisture-responsive composite according to any preceding Example, wherein the moisture-responsive polymer system further comprises a filler.

Example 50. The moisture-responsive composite according to any preceding Example, wherein the filler comprises fibers, particles, or a combination thereof.

Example 51. The moisture-responsive composite according to any preceding Example, wherein the filler comprises calcium carbonate particles, titanium dioxide particles, or a combination thereof.

Example 52. The moisture-responsive composite according to any preceding Example, further comprising a plasticizer.

Example 53. An absorbent article comprising the moisture-responsive composite according to any preceding Example.

Example 54. The absorbent article of any preceding Example, wherein the article is a diaper, training pants, swimwear, absorbent underpants, incontinence product, urinary shield, sanitary napkin, menstrual pad, panty liner, panty shield, interlabial, tampon, drape, gown, bandage, wound dressing, underpad, face mask, or bed pad.

Example 55. A method of making the moisture-responsive composite according to any preceding Example, comprising the steps: extruding a mixture comprising a hydrophilic polymer and elastomer to provide an unstretched extruded film; stretching the unstretched extruded film to provide a moisture-responsive polymer system; and laminating the moisture-responsive polymer system to a backing layer.

Example 56. The method according to any preceding Example, wherein the mixture is a pre-compounded mixture.

Example 57. The method according to any preceding Example, wherein the mixture is not a pre-compounded mixture.

Example 58. The method according to any preceding Example, wherein the mixture is extruded through a one screw extruder or a twin screw extruder.

Example 59. The method according to any preceding Example, wherein the stretching comprises stretching the unstretched extruded film in the machine direction.

Example 60. The method according to any preceding Example, comprising stretching the unstretched extruded film in the machine direction to give a first stretched extruded film, and stretching said first stretched extruded film to provide the moisture-responsive polymer system.

Example 61. The method according to any preceding Example, wherein the unstretched extruded film is stretched under different conditions than the first stretched extruded film.

Example 62. The method according to any preceding Example, wherein the unstretched extruded film is stretched in the machine direction, and the first stretched extruded film is stretched in the machine direction or a direction other than the machine direction.

Example 63. The moisture-responsive composite according to any preceding Example, wherein the first stretched extruded film is stretched in a direction that is from 1-30°, from 25-50°, from 30-60°, from 40-75°, from 50-80°, or from 60-90° from the machine direction.

Example 64. The moisture-responsive composite according to any preceding Example, wherein the first stretched extruded film is stretched in a direction that is 5°, 10°, 15°, 20°, 25°, 30°, 35°, 40°, 45°, 50°, 55°, 60°, 65°, 70°, 75°, 80°, 85°, or 90° from the machine direction.

Example 65. The method according to any preceding Example, wherein the unstretched extruded film is stretched at a temperature different than the temperature at which the first stretched extruded film is stretched.

Example 66. The method according to any preceding Example, wherein the unstretched extruded film is stretched at a temperature greater than the temperature at which the first stretched extruded film is stretched.

Example 67. The method according to any preceding Example, wherein the unstretched extruded film is stretched at a temperature below the temperature at which the first stretched extruded film is stretched.

Example 68. The method according to any preceding Example, wherein the unstretched extruded film is stretched using a stretching force that is different than the stretching force used to stretch the first stretched extruded film.

Example 69. The method according to any preceding Example, wherein the unstretched extruded film is stretched using a stretching force that is greater than the stretching force used to stretch the first stretched extruded film.

Example 70. The method according to any preceding Example, wherein the unstretched extruded film is stretched using a stretching force that is less than the stretching force used to stretch the first stretched extruded film.

Example 71. The method according to any preceding Example, wherein the unstretched extruded film is stretched at a temperature from 20-30° C., 20-150° C., from 50-150° C., from 75-125° C., or from 100-150° C.

Example 72. The method according to any preceding Example, wherein the unextruded film is stretched to a stretch ratio from 2-8, 2-6, 2-4, 3-6, 3-6, 4-6, 4-7, 5-7, or 5-8.

Example 73. The method according to any preceding Example, wherein the unstretched extruded film is stretched to a length that is 2-6 times, 2-4 times, 3-6 times, 3-6 times, 4-6 times, 4-7 times, 5-7 times, or 5-8 times, the length of the unstretched extruded film prior to stretching.

Example 74. The method according to any preceding Example, wherein the backing layer comprises nonwoven materials, films, wetlaid, airlaid, spunbond, meltblown, coform, bonded-carded webs, foams, tissue, netting, scrim, woven materials, or a combination thereof.

Example 75. The method according to any preceding Example, wherein the backing layer has a basis weight between about 1-1,000, grams per square meter, for example 1-50, 1-25, 1-10, 1-5, 5-10, 5-15, 10-20, 10-25, 10-50, or 25-50 grams per square meter.

Example 76. The method according to any preceding Example, wherein the backing layer has a basis weight between about 1-5,000, grams per square meter, for example about 1-1,000, about 50-1,000, about 100-1,000, about 1-500, about 1-250, about 5-250, about 10-250, about 10-100, about 50-100, about 75-125, about 100-150, about 50-150, about 100-250, about 250-500, or about 500-1,000 grams per square meter.

Example 77. The method according to any preceding Example, wherein the moisture-responsive polymer system is adhesively laminated to the backing layer.

Example 78. The method according to any preceding Example, wherein the moisture-responsive polymer system is adhesively laminated to the backing layer by a hot melt adhesive, a pressure-sensitive adhesive, or a combination thereof.

Example 79. A moisture sensitive composite, prepared by a method according to any preceding Example.

Example 80. A moisture-responsive polymer system, comprising a stretched polymer film comprising a hydrophilic polymer and an elastomer.

Example 81. The moisture-responsive polymer system according to any preceding Example, having a basis weight between about 1-5,000, about 1-1,000, about 50-1,000, about 100-1,000, about 1-500, about 1-250, about 5-250, about 10-250, about 10-100, about 10-50, about 25-50, about 25-75, about 50-100, about 75-125, about 100-150, about 50-150, about 100-250, about 250-500, or about 500-1,000 grams per square meter.

Example 82. The moisture-responsive polymer system according to any preceding Example, having a stretch ratio from 2-6, 2-4, 3-6, 3-6, 4-6, 4-7, 5-7, or 5-8.

Example 83. The moisture-responsive polymer system according to any preceding Example, wherein the elongated moisture-responsive polymer system is stretched to a length that is 2-6 times, 2-4 times, 3-6 times, 3-6 times, 4-6 times, 4-7 times, 5-7 times, or 5-8 times the length of the moisture-responsive polymer system prior to stretching.

Example 84. The moisture-responsive polymer system according to any preceding Example, having a basis weight (gsm) that is no more than 90%, no more than 80%, no more than 70%, no more than 60%, no more than 50%, no more than 40%, no more than 30%, or no more than 20% the basis weight of the moisture-responsive polymer system prior to stretching.

Example 85. The moisture-responsive polymer system according to any preceding Example, having a width that is no more than 90%, no more than 80%, no more than 70%, no more than 60%, no more than 50%, no more than 40%, no more than 30%, or no more than 20% the width of the moisture-responsive polymer system prior to stretching.

Example 86. The moisture-responsive polymer system according to any preceding Example, comprising a hydrophilic polymer and an elastomer in a wt. ratio (hydrophilic polymer:elastomer) from: no more than 75 wt. % hydrophilic polymer, e.g., from 25:75 to 75:25, from 30:70 to 75:25, from 35:65 to 75:25, from 40:60 to 75:25, from 45:55 to 75:25, from 50:50 to 75:25, from 55:45 to 75:25, from 60:40 to 75:25, from 65:35 to 75:25; no more than 70 wt. % hydrophilic polymer, e.g., from 25:75 to 70:30, from 30:70 to 70:30, from 35:65 to 70:30, from 40:60 to 70:30, from 45:55 to 70:30, from 50:50 to 70:30, from 55:45 to 70:30, from 60:40 to 70:30, from 65:35 to 70:30; no more than 65 wt. % hydrophilic polymer, e.g., from 25:75 to 65:35, from 30:70 to 65:35, from 35:65 to 65:35, from 40:60 to 65:35, from 45:55 to 65:35, from 50:50 to 65:35, from 55:45 to 65:35, from 60:30 to 65:35; no more than 60 wt. % hydrophilic polymer, e.g., from 25:75 to 60:40, from 30:70 to 60:40, from 35:65 to 60:40, from 40:60 to 60:40, from 45:55 to 60:40, from 50:50 to 60:40, from 55:45 to 60:40; no more than 55 wt. % hydrophilic polymer, e.g., from 25:75 to 55:45, from 30:70 to 55:45, from 35:65 to 55:45, from 40:60 to 55:45, from 45:55 to 55:45, from 50:50 to 55:45; no more than 50 wt. % hydrophilic polymer, e.g., from 25:75 to 50:50, from 30:70 to 50:50, from 35:65 to 50:50, from 40:60 to 50:50, from 45:55 to 50:50; no more than 45 wt. % hydrophilic polymer, e.g., from 25:75 to 45:55, from 30:70 to 45:55, from 35:65 to 45:55, from 40:60 to 45:55; no more than 40 wt. % hydrophilic polymer, e.g., from 25:75 to 40:60, from 30:70 to 40:60, from 35:65 to 40:60; or no more than 35 wt. % hydrophilic polymer, e.g., from 25:75 to 35:65, from 30:70 to 35:65.

Example 87. The moisture-responsive polymer system according to any preceding Example, comprising a hydrophilic polymer and an elastomer in a wt. ratio (hydrophilic polymer:elastomer) from 70:30 to 50:50.

Example 88. The moisture-responsive polymer system according to any preceding Example, comprising a hydrophilic polymer and an elastomer in a wt. ratio (hydrophilic polymer:elastomer) from 70:30 to 55:45.

Example 89. The moisture-responsive polymer system according to any preceding Example, comprising a hydrophilic polymer and an elastomer in a wt. ratio (hydrophilic polymer:elastomer) from 70:30 to 60:40.

Example 90. The moisture-responsive polymer system according to any preceding Example, comprising a hydrophilic polymer and an elastomer in a wt. ratio (hydrophilic polymer:elastomer) from 60:40 to 50:50.

Example 91. The moisture-responsive polymer system according to any preceding Example, comprising a hydrophilic polymer having a MW from 10,000-1,000,000 Da, from 10,000-500,000 Da, from 50,000-500,000 Da, from 50,000-250,000 Da, from 50,000-1000,000, from 100,000-500,000 Da, from 100,000-300,000 Da, from 200,000-400,000 Da, from 150,000-300,000 Da, or from 250,000-500,000 Da.

Example 92. The moisture-responsive polymer system according to any preceding Example, comprising a poly(vinyl pyrrolidone), poly(hydroxyethyl (meth)acrylate, poly(hydroxypropyl (meth)acrylate, poly(meth)acrylic acid, poly(vinyl pyridine), poly(meth)acrylamide, poly(vinyl acetate), poly(vinyl alcohol), poly(ethylene oxide), or a combination thereof.

Example 93. The moisture-responsive polymer system according to any preceding Example, comprising poly(ethylene oxide).

Example 94. The moisture-responsive polymer system according to any preceding Example, comprising a poly(ethylene oxide) homopolymer, a poly(ethylene oxide-co-propylene oxide) block copolymer, or a combination thereof.

Example 95. The moisture-responsive polymer system according to any preceding Example, having a solubility in water, at 23° C., of at least 0.1 g/mL, at least 0.5 g/mL, at least 1 g/mL, at least 2.5 g/mL, at least 5 g/mL, or at least 10 g/mL.

Example 96. The moisture-responsive polymer system according to any preceding Example, having a degree of crystallinity of at least 25%, at least 30%, at least 40%, at least 50%, at least 60%, at least 70%, at least 80%, or at least 90%.

Example 97. The moisture-responsive polymer system according to any preceding Example, comprising a degree of crystallinity of at least 25%, at least 30%, at least 40%, at least 50%, at least 60%, at least 70%, at least 80%, or at least 90%, and when submerged in water, converts to at least 90% amorphous form within 120 seconds, 100 seconds, 80 seconds, 60 seconds, 50 seconds, 40 seconds, 30 seconds, 20 seconds, or at least 10 seconds.

Example 98. The moisture-responsive polymer system according to any preceding Example, comprising an elastomer having a MW_{w} from 1,000-1,000,000 Da, from 1,000-10,000 Da, from 1,000-5,000 Da, from 2,500-10,000 Da, from 2,500-7,500 Da, from 2,500-10,000 Da, from 5,000-10,000 Da, from 5,000-25,000 Da, from 10,000-20,000 Da, from 10,000-50,000 Da, from 10,000-100,000, from 10,000-250,000 Da, from 25,000-250,000 Da, from 50,000-250,000 Da, from 50,000-100,000 Da, from 100,000-250,000 Da, from 10,000-500,000 Da, from 50,000-500,000 Da, from 100,000-500,000 Da, from 100,000-300,000 Da, from 200,000-400,000 Da, from 150,000-300,000 Da, or from 250,000-500,000 Da.

Example 99. The moisture-responsive polymer system according to any preceding Example, comprising a polyolefin elastomer.

Example 100. The moisture-responsive polymer system according to any preceding Example, comprising a styrene block copolymer, a butadiene block copolymer, a hydrogenated butadiene block copolymer, an isoprene block copolymer, a hydrogenated isoprene block copolymer, non-crystalline ethylene/α-olefin random copolymer, low-crystalline ethylene/α-olefin random copolymer, propylene/ethylene/α-olefin random copolymer, or combination thereof.

Example 101. The moisture-responsive polymer system according to any preceding Example, comprising a polystyrene-polybutadiene-polystyrene block copolymer (SBS), polystyrene-polyisoprene-polystyrene block copolymer (SIS), polystyrene-poly/ethylene/butylene-polystyrene block copolymer (SEBS), polystyrene-poly/ethylene/propylene-polystyrene block copolymer, or a combination thereof.

Example 102. The moisture-responsive polymer system according to any preceding Example, comprising a polyolefin elastomer comprising an ethylene/propylene random copolymer, ethylene/1-butene random copolymer, propylene/1-butene random copolymer, or combination thereof.

Example 103. The moisture-responsive polymer system according to any preceding Example, comprising a polyester elastomer, polyamide elastomer, or a combination thereof.

Example 104. The moisture-responsive polymer system according to any preceding Example, comprising an ethylene/α-olefin copolymer, propylene/α-olefin copolymer, styrene-olefin copolymer, or a combination thereof.

Example 105. The moisture-responsive composite according to any preceding Example, wherein the elastomer comprises poly(ethylene-butene), poly(ethylene-hexene), poly(ethylene-octene), poly(ethylene-propylene), poly(styrene-butadiene-styrene), poly(styrene-isoprene-styrene), poly(styrene-ethylene-butylene-styrene), poly(ester-ether), poly(ether-amide), poly(ethylene-vinylacetate), poly(ethylene-methylacrylate), poly(ethylene-acrylic acid), poly(ethylene butylacrylate), polyurethane, poly(ethylene-propylene-diene), ethylene-propylene rubber, or a combination thereof.

Example 106. The moisture-responsive polymer system according to any preceding Example, further comprising a filler.

Example 107. The moisture-responsive polymer system according to any preceding Example, comprising a filler comprising fibers, particles, or a combination thereof.

Example 108. The moisture-responsive polymer system according to any preceding Example, comprising calcium carbonate particles, titanium dioxide particles, or a combination thereof.

Example 109. The moisture-responsive polymer system according to any preceding Example, further comprising a plasticizer.

Example 110. An absorbent article comprising the moisture-responsive polymer system according to any preceding Example.

Example 111. The absorbent article of any preceding Example, wherein the article is a diaper, training pants, swimwear, absorbent underpants, incontinence product, urinary shield, sanitary napkin, menstrual pad, panty liner, panty shield, interlabial, tampon, drape, gown, bandage, wound dressing, underpad, face mask, or bed pad.

Example 112. A method of making the moisture-responsive polymer system according to any preceding Example, comprising the steps: extruding a mixture comprising a hydrophilic polymer and elastomer to provide an unstretched extruded film; stretching the unstretched extruded film to provide a moisture-responsive polymer system.

Example 113. The method according to any preceding Example, wherein the mixture is a pre-compounded mixture.

Example 114. The method according to any preceding Example, wherein the mixture is not a pre-compounded mixture.

Example 115. The method according to any preceding Example, wherein the mixture is extruded through a one screw extruder or a twin screw extruder.

Example 116. The method according to any preceding Example, wherein the stretching comprises stretching the unstretched extruded film in the machine direction.

Example 117. The method according to any preceding Example, comprising stretching the unstretched extruded film in the machine direction to give a first stretched extruded film, and stretching said first stretched extruded film to provide the moisture-responsive polymer system.

Example 118. The method according to any preceding Example, wherein the unstretched extruded film is stretched under different conditions than the first stretched extruded film.

Example 119. The method according to any preceding Example, wherein the unstretched extruded film is stretched in the machine direction, and the first stretched extruded film is stretched in the machine direction or a direction other than the machine direction.

Example 120. The moisture-responsive composite according to any preceding Example, wherein the first stretched extruded film is stretched in a direction that is from 1-30°, from 25-50°, from 30-60°, from 40-75°, from 50-80°, or from 60-90° from the machine direction.

Example 121. The moisture-responsive composite according to any preceding Example, wherein the first stretched extruded film is stretched in a direction that is 5°, 10°, 15°, 20°, 25°, 30°, 35°, 40°, 45°, 50°, 55°, 60°, 65°, 70°, 75°, 80°, 85°, or 90° from the machine direction.

Example 122. The method according to any preceding Example, wherein the unstretched extruded film is stretched at a temperature different than the temperature at which the first stretched extruded film is stretched.

Example 123. The method according to any preceding Example, wherein the unstretched extruded film is stretched at a temperature greater than the temperature at which the first stretched extruded film is stretched.

Example 124. The method according to any preceding Example, wherein the unstretched extruded film is stretched at a temperature below the temperature at which the first stretched extruded film is stretched.

Example 125. The method according to any preceding Example, wherein the unstretched extruded film is stretched using a stretching force that is different than the stretching force used to stretch the first stretched extruded film.

Example 126. The method according to any preceding Example, wherein the unstretched extruded film is stretched using a stretching force that is greater than the stretching force used to stretch the first stretched extruded film.

Example 127. The method according to any preceding Example, wherein the unstretched extruded film is stretched using a stretching force that is less than the stretching force used to stretch the first stretched extruded film.

Example 128. The method according to any preceding Example, wherein the unstretched extruded film is stretched at a temperature from 20-30° C., 20-150° C., from 50-150° C., from 75-125° C., or from 100-150° C.

Example 129. The method according to any preceding Example, wherein the unextruded film is stretched to a stretch ratio from 2-6, 2-4, 3-6, 3-6, 4-6, 4-7, 5-7, or 5-8.

Example 130. The method according to any preceding Example, wherein the unstretched extruded film is stretched to a length that is 2-6 times, 2-4 times, 3-6 times, 3-6 times, 4-6 times, 4-7 times, 5-7 times, or 5-8 times the length of the unstretched extruded film prior to stretching.

Example 131. A moisture-responsive polymer system, prepared by a method according to any preceding Example.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, 58epree only certain 58epressentative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A moisture-responsive composite comprising:
a moisture-responsive layer comprising a moisture-responsive polymer system; and
a backing layer laminated with the moisture-responsive layer;
wherein, upon exposure to moisture, the moisture-responsive composite bends from a first position to a second position.

2. The moisture-responsive composite of claim 1, wherein the moisture-responsive layer is elongated and defines a longitudinal axis; and
wherein the moisture-responsive composite's longitudinal axis defines a radius of curvature that decreases as the moisture-responsive composite bends from the first position to the second position.

3. The moisture-responsive composite of claim 2, wherein the movement from the first position towards the second position decreases the radius of curvature of the center line by at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

4. The moisture-responsive composite of claim 1, wherein the backing layer has a higher elastic modulus than the moisture-responsive layer.

5. The moisture-responsive composite of claim 1, wherein the backing layer has a higher Young's modulus than the moisture-responsive layer.

6. The moisture-responsive composite of claim 1, wherein the backing layer has a higher bending stiffness than the moisture-responsive layer.

7. The moisture-responsive composite of claim 2, wherein the elongated moisture-responsive polymer system is stretched to a length that is 2-6 times, 2-4 times, 3-6 times, 3-6 times, 3-7 times, or 3-8 times the length of the moisture-responsive polymer system prior to stretching.

8. The moisture-responsive composite of claim 2, wherein the elongated moisture-responsive polymer system has a basis weight (gsm) that is no more than 90%, no more than 80%, no more than 70%, no more than 60%, no more than 50%, no more than 40%, or no more than 30% the basis weight of the moisture-responsive polymer system prior to stretching.

9. The moisture-responsive composite of claim 1, wherein the moisture-responsive polymer system comprises a hydrophilic polymer and an elastomer.

10. The moisture-responsive composite of claim 1, wherein the moisture-responsive polymer system comprises a hydrophilic polymer having a MW_{w} from 10,000-1,000,000 Da, from 10,000-500,000 Da, from 50,000-500,000 Da, from 50,000-250,000 Da, from 50,000-1000,000, from 100,000-500,000 Da, from 100,000-300,000 Da, from 200,000-400,000 Da, from 150,000-300,000 Da, or from 250,000-500,000 Da.

11. The moisture-responsive composite of claim 1, wherein the moisture-responsive polymer system comprises a poly(vinyl pyrrolidone), poly(hydroxyethyl (meth)acrylate, poly(hydroxypropyl (meth)acrylate, poly(meth)acrylic acid, poly(vinyl pyridine), poly(meth)acrylamide, poly(vinyl acetate), poly(vinyl alcohol), poly(ethylene oxide), or a combination thereof.

12. The moisture-responsive composite of claim 1, wherein the moisture-responsive polymer system comprises poly(ethylene oxide).

13. The moisture-responsive composite of claim 9, wherein when the moisture-responsive composite is in the first position, the hydrophilic polymer has a degree of crystallinity of at least 25%, at least 30%, at least 40%, at least 50%, at least 60%, at least 70%, at least 80%, or at least 90%, and when submerged in water, converts to at least 90% amorphous form within 120 seconds, 100 seconds, 80 seconds, 60 seconds, 50 seconds, 40 seconds, 30 seconds, 20 seconds, or at least 10 seconds.

14. The moisture-responsive composite of claim 1, wherein the moisture-responsive polymer system comprises an elastomer having a MW_{w} from 1,000-1,000,000 Da, from 1,000-10,000 Da, from 1,000-5,000 Da, from 2,500-10,000 Da, from 2,500-7,500 Da, from 2,500-10,000 Da, from 5,000-10,000 Da, from 5,000-25,000 Da, from 10,000-20,000 Da, from 10,000-50,000 Da, from 10,000-100,000, from 10,000-250,000 Da, from 25,000-250,000 Da, from 50,000-250,000 Da, from 50,000-100,000 Da, from 100,000-250,000 Da, from 10,000-500,000 Da, from 50,000-500,000 Da, from 100,000-500,000 Da, from 100,000-300,000 Da, from 200,000-400,000 Da, from 150,000-300,000 Da, or from 250,000-500,000 Da.

15. The moisture-responsive composite of claim 14, wherein the elastomer comprises a styrene block copolymer, a butadiene block copolymer, a hydrogenated butadiene block copolymer, an isoprene block copolymer, a hydrogenated isoprene block copolymer, non-crystalline ethylene/α-olefin random copolymer, low-crystalline ethylene/α-olefin random copolymer, propylene/ethylene/α-olefin random copolymer, or combination thereof.

16. The moisture-responsive composite of claim 14, wherein the elastomer comprises poly(ethylene-butene), poly(ethylene-hexene), poly(ethylene-octene), poly(ethylene-propylene), poly(styrene-butadiene-styrene), poly(styrene-isoprene-styrene), poly(styrene-ethylene-butylene-styrene), poly(ester-ether), poly(ether-amide), poly(ethylene-vinylacetate), poly(ethylene-methylacrylate), poly(ethylene-acrylic acid), poly(ethylene butylacrylate), polyurethane, poly(ethylene-propylene-diene), ethylene-propylene rubber, or a combination thereof.

17. The moisture-responsive composite of claim 1, wherein the backing layer comprises a hydrophobic material, a hydrophilic material, or a combination thereof.

18. The moisture-responsive composite of claim 1, wherein the backing layer comprises nonwoven materials, films, wetlaid, airlaid, spunbond, meltblown, coform, bonded-carded webs, foams, tissue, netting, scrim, woven materials, or a combination thereof.

19. The moisture-responsive composite of claim 1, wherein the moisture-responsive layer is adhesively laminated or autogenously laminated to the backing layer.

20. An absorbent article comprising the moisture-responsive composite of claim 1.
